(19) United States
(12) Patent Application Publication
(10) Pub. No.: US 2010/0056575 A1
(43) Pub. Date: Mar. 4, 2010

(54) Anesthetic Compounds

(76) Inventor: Thomas E. Jenkins, Half Moon Bay, CA (US)

Correspondence Address: PharmacoFore, Inc.;Bozicevic, Field & Francis LLP, 1900 University Avenue, Suite 200, East Palo Alto, CA, 94303, US

(21) Appl. No.: 12/373,717

(22) PCT Filed: Jul. 19, 2007
(86) PCT No.: PCT/US07/73926

§ 371 Date: Nov. 13, 2009

Related U.S. Application Data
(60) Provisional application No. 60/832,174, filed on Jul. 19, 2006.
(60) Provisional application No. 60/832,694, filed on Jul. 20, 2006.
(60) Provisional application No. 60/837,697, filed on Aug. 14, 2006.

(51) Int. Cl. A61K 31/4453; C07D 211/32; C07C 229/60; A61K 31/245; A61P 41/00

(52) U.S. Cl. 514/331; 546/235; 560/46; 514/535

(57) ABSTRACT

In one embodiment the invention provides novel compounds of Formula (I) as well as prodrugs, salts, hydrates, solvates and N-oxides thereof. The invention also provides pharmaceutical compositions that include such compounds as well as methods for making and methods for using such compounds in medical therapy.

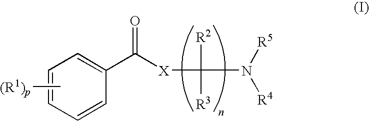

This application claims priority to U.S. Provisional Application Nos. 60/832,174, filed 19 Jul. 2006; 60/832,694, filed 20 Jul. 2006; and 60/837,697, filed 14 Aug. 2006.

Local anesthetics produce loss of sensation by binding to sodium channels and inhibiting sodium currents which causes blockade of sodium channel dependent impulse conduction. The action of local anesthetics is reversible at clinically relevant concentrations thus allowing for complete recovery of nerve and muscle function without damage to nerve fibers or cells.

Many clinically useful local anesthetics are comprised of a substituted aromatic group attached to a carboxylic acid derivative such as an amide or ester to which is connected a secondary or tertiary amino group via an alkyl linker. Ester anesthetics, whose clinical use was first discovered at the beginning of the 20^{th} century include, for example, cocaine, procaine, tetracaine, benzocaine, amethocaine and chloroprocaine. Amide anesthetics, which were first clinically used prior to the Second World War include, for example, lidocaine, prilocaine, mepivacaine, ropivacaine, etidocaine, levobupivacaine and bupivacaine. Despite being discovered first, the use of ester anesthetics has been largely supplanted by amide anesthetics.

Notwithstanding the chemical similarities between these two classes of local anesthetics, clinically important differences exist between amide and ester anesthetics. Significantly all local anesthetics share similar toxicity profiles (e.g., seizures from central nervous system toxicity, arrhythmia and death from cardiac toxicity) which suggests that the rate of metabolism may be an important factor in fatalities caused by anesthetics. Ester anesthetics are rapidly hydrolyzed in vivo by plasma cholinesterases while amide anesthetics are hydrolyzed much less rapidly by hepatic proteases. Rapid metabolism prevents esters from reaching toxic level in vivo even with large or repeated doses. Amide anesthetics, in contrast, can accumulate to toxic levels with large or repeated dosages because of slow hydrolysis in vivo.

Another important issue with currently used local anesthetics is the limited duration of action which is often too short to relieve post-operative pain, slow onset of action which limits utility in postoperative settings in addition to aforementioned safety issues. Further, many currently used anesthetics cause pain and discomfort when administered to a patient.

Accordingly, in view of the foregoing, what is needed are local anesthetics which have rapid onset, longer duration of action, and/or minimal side effects.

The present invention satisfies these and other needs by providing novel ester local anesthetics. Also disclosed herein are methods of making novel ester local anesthetics, pharmaceutical compositions of novel ester local anesthetics and methods of using novel ester local anesthetics and pharmaceutical compositions thereof to induce and/or maintain anesthesia and/or analgesia.

In one embodiment the invention provides a compound of the invention that is of structural Formula (I):

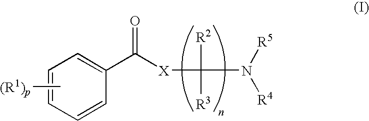

or a prodrug, salt, hydrate, solvate or N-oxide thereof wherein:

each R¹ is independently hydrogen, —F, —Cl, —Br, —I, —R⁶, —OR⁶, —SR⁶, —NR⁶R⁷ or —C(O)NR⁶R⁷,—provided that at least one R¹ is R⁶, —OR⁶, —SR⁶, —NR⁶R⁷ or —C(O)NR⁶R;

p is an integer from 1 to 3;

X is —O— or —S—;

R² and R³ are independently hydrogen or (C1-C6) alkyl optionally substituted with one or more of the same or different R⁸ groups or optionally one of either R² or R³ and one of either R⁴ or R⁵ together with the atoms to which they are bonded form a cycloheteroalkyl ring; or R² is the sidechain of an amino acid and R³ is H;

n is 1, 2, 3, or 4;

R⁴ and R⁵ are independently H, (C1-C6) alkyl optionally substituted with one or more of the same or different R⁸ groups or optionally R⁴ and R⁵ together with the nitrogen atom to which they are bonded form a cycloheteroalkyl ring that is optionally substituted with one or more (C1-C6) alkyl;

R⁶ and R⁷ are independently hydrogen, alkyl optionally substituted with one or more of the same or different R⁸ groups, cycloalkyl optionally substituted with one or more of the same or different R⁸ groups, cycloheteroalkyl optionally substituted with one or more of the same or different R⁸ groups, aryl optionally substituted with one or more of the same or different R⁸ groups or heteroaryl optionally substituted with one or more of the same or different R⁸ groups; or optionally R⁶ and R⁷ together with the nitrogen atom to which they are bonded form a cycloheteroalkyl ring and

R⁸ is —F, —Cl, —Br, —I, —R⁶, —OR⁶, —SR⁶, —NR⁶R⁷, —CF₃, —CN, —C(O)R⁶, —C(O)OR⁶, —C(O)SR⁶, —C(O)NR⁶R⁷, or aryl which is optionally substituted with one or more —F, —Cl, —Br, —I, —R⁶, —OR⁶, —SR⁶, —NR⁶R⁷, —CF₃, —CN, —C(O)R⁶, —C(O)OR⁶, —C(O)SR⁶, or —C(O)NR⁶R⁷.

In one embodiment the compounds of formula I exclude the following structures:

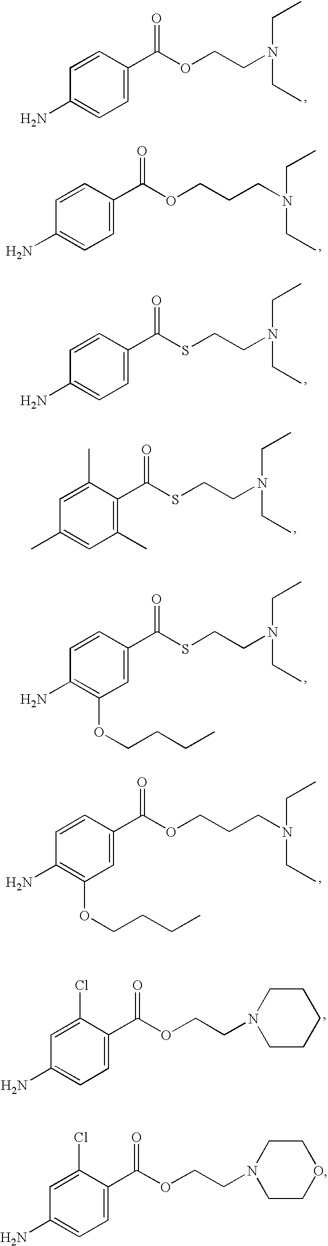

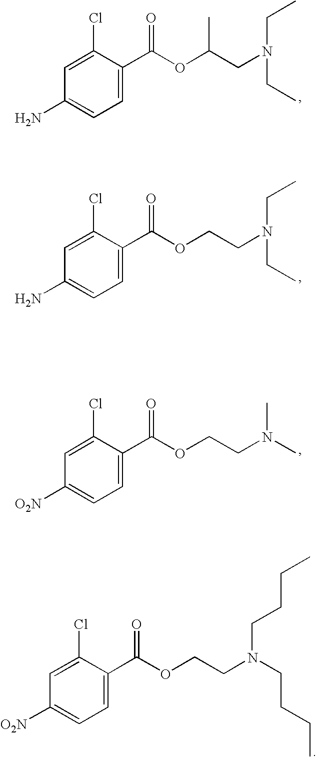

In another embodiment the invention provides a method for inducing and/or maintaining anesthesia and/or analgesia comprising administering to a patient in need of such treatment or prevention a therapeutically effective amount of a compound of formula (I) or a prodrug, salt, hydrate, solvate or N-oxide thereof.

In another embodiment the invention provides a pharmaceutical composition comprising a compound of formula (I) or a prodrug, salt, hydrate, solvate or N-oxide thereof, and a pharmaceutically acceptable vehicle.

In another embodiment the invention provides a method of inducing or maintaining local anesthesia in a patient comprising administering to the patient a compound of formula (I) or a prodrug, salt, hydrate, solvate or N-oxide thereof.

In another embodiment the invention provides a method of treating or preventing pain in a patient comprising administering to the patient in need thereof a compound of formula (I) or a prodrug, salt, hydrate, solvate or N-oxide thereof.

In another embodiment the invention provides a compound of formula I, or a prodrug, salt, hydrate, solvate or N-oxide thereof for use in medical therapy.

In another embodiment the invention provides the use of a compound of formula I, or a prodrug, salt, hydrate, solvate or N-oxide thereof to prepare a medicament for inducing or maintaining local anesthesia in a mammal such as a human.

In another embodiment the invention provides the use of a compound of formula I, or a prodrug, salt, hydrate, solvate or N-oxide thereof to prepare a medicament for treating or preventing pain in a mammal such as a human.

The invention also provides novel processes and synthetic intermediates disclosed herein that are useful for preparing a compound of formula I, or a prodrug, salt, hydrate, solvate or N-oxide thereof. Some compounds of formula I may be useful as intermediates for preparing other compounds of formula I.

### DETAILED DESCRIPTION

### Definitions

“Alkyl” by itself or as part of another substituent refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms.

“Alkanyl” by itself or as part of another substituent refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Amino Acid,” refers to the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine).

“Compounds” as used herein refers to compounds encompassed by structural Formula (I) and disclosed herein and includes any specific compounds within this formula whose structure is disclosed herein. Compounds may be identified either by their chemical structure and/or chemical name. The compounds described herein may contain one or more chiral centers and/or double bonds and therefore, may exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds.

“Aryl” as used herein refers to a phenyl radical or an ortho-fused bicyclic carbocyclic radical having about nine to ten ring atoms in which at least one ring is aromatic. Examples of aryl include phenyl, indenyl, and naphthyl.

“Heteroaryl” as used herein refers to a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) and quinolyl (or its N-oxide).

“Cycloalkyl” as used herein refers to a cyclic “alkyl” group.

The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc.

Compounds may exist in unsolvated forms as well as solvated forms, including hydrated forms. Certain compounds may exist in multiple crystalline or amorphous forms. In general, all physical forms are within the scope of the present disclosure.

“Cycloheteroalkyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Pharmaceutical composition” refers to at least one compound and a pharmaceutically acceptable vehicle.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound is administered.

“Patient” includes mammals, such as humans. The terms “human” and “patient” are used interchangeably herein.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Therapeutically effective amount” means the amount of a compound that, when administered to a patient for inducing and/or maintaining anesthesia or for providing analgesia, is sufficient to effect such induction or maintenance of anesthesia and/or analgesia. The “therapeutically effective amount” will vary depending on the compound, the disease and its severity, and the age, weight, etc., of the patient to be treated.

Reference will now be made in detail to various embodiments. It will be understood that the invention is not limited to these embodiments. To the contrary, it is intended to cover alternatives, modifications, and equivalents as may be included within the spirit and scope of the allowed claims.

In one embodiment a compound of structural Formula (I) is provided:

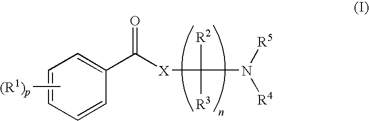

or prodrugs, salts, hydrates, solvates or N-oxides thereof wherein:

each R¹ is independently hydrogen, —F, —Cl, —Br, —I, —R⁶, —OR⁶, —SR⁶, —NR⁶R⁷ or —C(O)NR⁶R⁷,—provided that at least one R¹ is R⁶, —OR⁶, —SR⁶, —NR⁶R⁷ or —C(O)NR⁶R;

p is an integer from 1 to 3;

X is —O— or —S—;

R² and R³ are independently hydrogen or (C1-C6) alkyl optionally substituted with one or more of the same or different R⁸ groups or optionally one of either R² or R³ and one of either R⁴ or R⁵ together with the atoms to which they are bonded form a cycloheteroalkyl ring;

n is an integer from 0 to 4;

R⁴ and R⁵ are independently (C1-C6) alkyl optionally substituted with one or more of the same or different R⁸ groups or optionally R⁴ and R⁵ together with the nitrogen atom to which they are bonded form a cycloheteroalkyl ring;

R⁶ and R⁷ are independently hydrogen, alkyl optionally substituted with one or more of the same or different R⁸ groups, cycloalkyl optionally substituted with one or more of the same or different R⁸ groups, cycloheteroalkyl optionally substituted with one or more of the same or different R⁸ groups, aryl optionally substituted with one or more of the same or different R⁸ groups or heteroaryl optionally substituted with one or more of the same or different R⁸ groups; and

R⁸ is —F, —Cl, —Br, —I, —R⁶, —OR⁶, —SR⁶, —NR⁶R⁷, —CF₃, —CN, —C(O)R⁶, —C(O)OR⁶, —C(O)SR⁶ or —C(O)NR⁶R⁷.

In some embodiments, R⁴ and R⁵ form only one cycloheteroalkyl ring. In other embodiments, R¹ is hydrogen, —Cl, —R⁶, —OR⁶ or —NR⁶R⁷, R² and R³ are hydrogen or (C1-C6) alkyl and R⁴ and R⁵ are (C1-C6) alkyl. In still other embodiments, R¹ is hydrogen, —Cl, —R⁶, —OR⁶ or —NR⁶R⁷. In still other embodiments, R² and R³ are hydrogen or (C1-C6) alkyl. In still other embodiments, R⁴ and R⁵ are (C1-C6) alkyl. In still other embodiments, R⁸ is —F, —Cl, —R⁶, —OR⁶, —SR⁶, —NR⁶R⁷, —C(O)R⁶, —C(O)OR⁶ or —C(O)NR⁶R⁷.

In some embodiments, p is 2, R¹ is —Cl and —NH₂, X is O, n is 2, R² and R³ are hydrogen, and R⁴ and R⁵ together with the nitrogen atom to which they are bonded form a cycloheteroalkyl ring. In other embodiments, a compound having the structure below is provided:

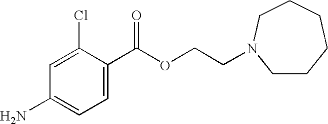

In some embodiments, p is 2, R¹ is —Cl and —NH₂, X is O, n is 2, each R³ is hydrogen, R² is hydrogen and R² and R⁴ together with the atoms to which they are bonded form a cycloheteroalkyl ring and R⁵ is hydrogen or (C1-C6) alkyl. In other embodiments compounds having the structures below are provided:

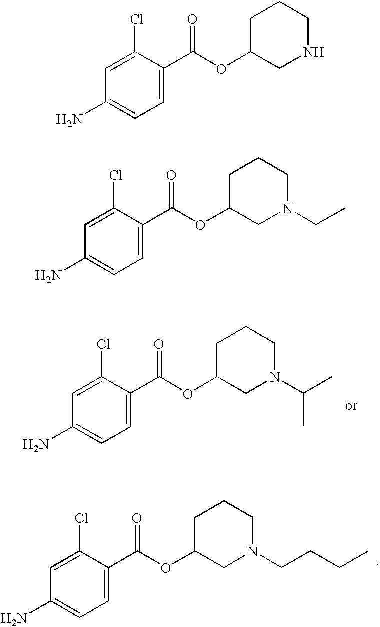

In other embodiments, compounds having the structures below are provided:

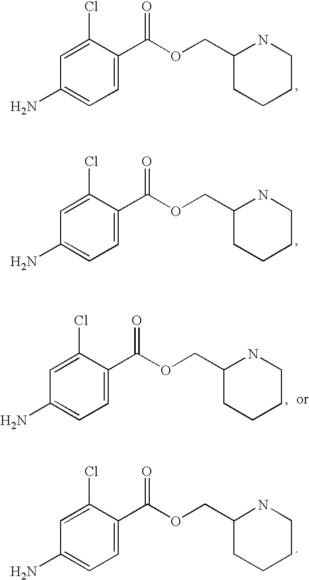

In still other embodiments, compounds having the structures below are provided:

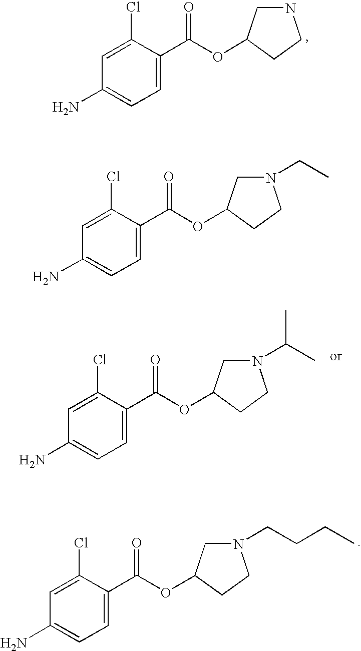

In some embodiments, p is 2, R¹ is —Cl and —NH₂, X is O, n is 3, R³ is hydrogen, R² is hydrogen and R² and R⁴ together with the atoms to which they are bonded form a cycloheteroalkyl ring and R⁵ is hydrogen or (C1-C6) alkyl. In still other embodiments, compounds having the structures below are provided:

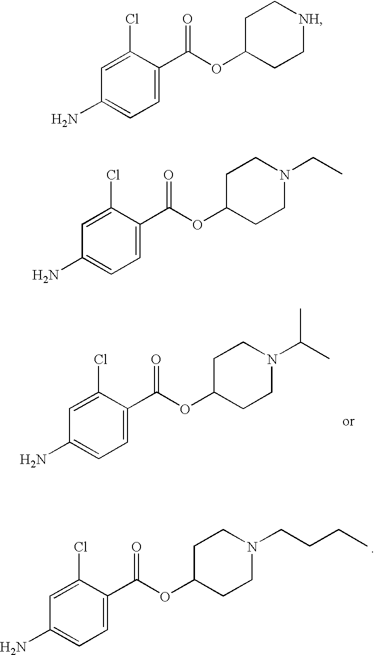

In some embodiments, p is 2, R¹ is —Cl and —NH₂, X is O, n is 4, R³ is hydrogen, R² is hydrogen and one R² and R⁴ together with the atoms to which they are bonded form a cycloheteroalkyl ring and R⁵ is hydrogen or (C1-C6) alkyl. In other embodiments, compounds having the structures below are provided:

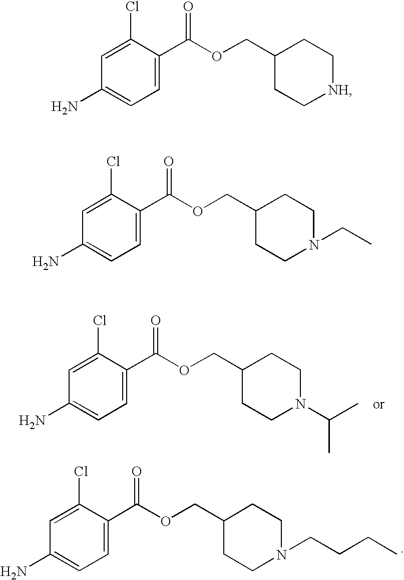

In some embodiments, p is 2, R¹ is OR⁶ and —NH₂, X is O, n is 2, R² and R³ are hydrogen and R⁵ is (C1-C6) alkyl. In other embodiments, a compound having the structure below is provided:

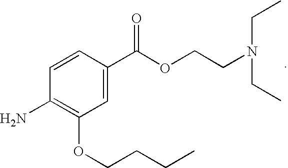

In some embodiments, p is 3, each R¹ is (C1-C6) alkyl, X is O, n is 2, R² and R³ are hydrogen and R⁵ is (C1-C6) alkyl. In other embodiments, a compound having the structure below is provided:

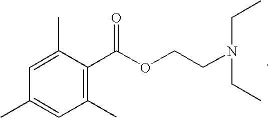

In some embodiments, R¹ is (C1-C6) alkyl, OR⁶ or —NH₂, X is S, n is 2, R² and R³ are hydrogen and R⁵ is (C1-C6) alkyl. In other embodiments, a compound having the structure below is provided:

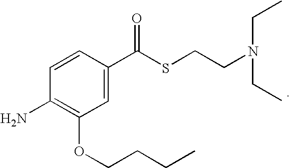

In some embodiments, R¹ is (C1-C6) alkyl, OR⁶ or —NH₂, X is O, n is 3, R² and R³ are hydrogen and R⁵ is (C1-C6) alkyl. In other embodiments, a compound having the structure below is provided:

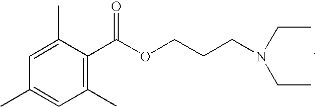

In some embodiments, R¹ is (C1-C6) alkyl, OR⁶ or —NH₂, X is O, n is 2, R² is hydrogen, R³ is hydrogen or (C1-C6) alkyl and R⁴ and R⁵ are (C1-C6) alkyl. In other embodiments, compounds having the following structures below are provided:

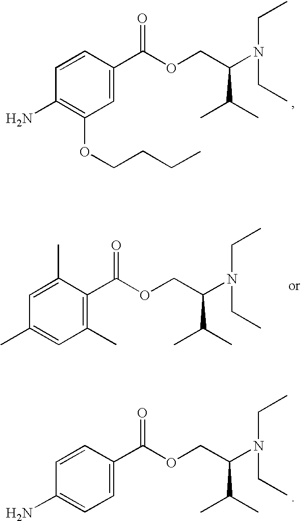

In some embodiments, R¹ is (C1-C6) alkyl, OR⁶ or —NH₂, X is O, n is 2, R² and R³ are hydrogen. In other embodiments, compounds having the structures below are provided:

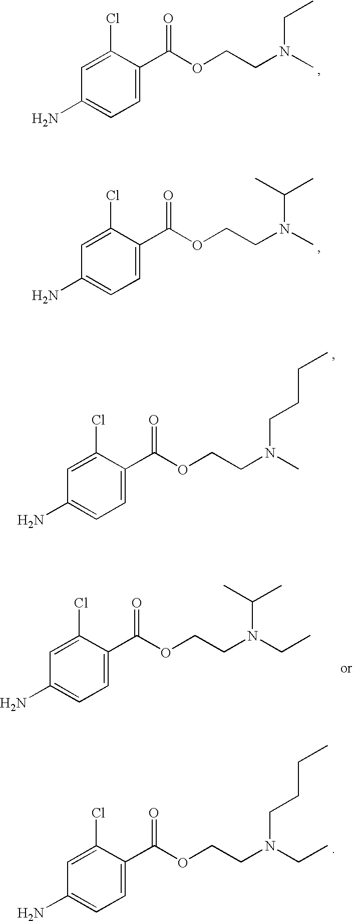

In some embodiments, R¹ is (C1-C6) alkyl, OR⁶ or —NH₂, X is O, n is 2, R² is hydrogen, R³ is hydrogen or (C1-C6) alkyl and R⁴ and R⁵ are (C1-C6) alkyl. In other embodiments, compounds having the structure below are provided:

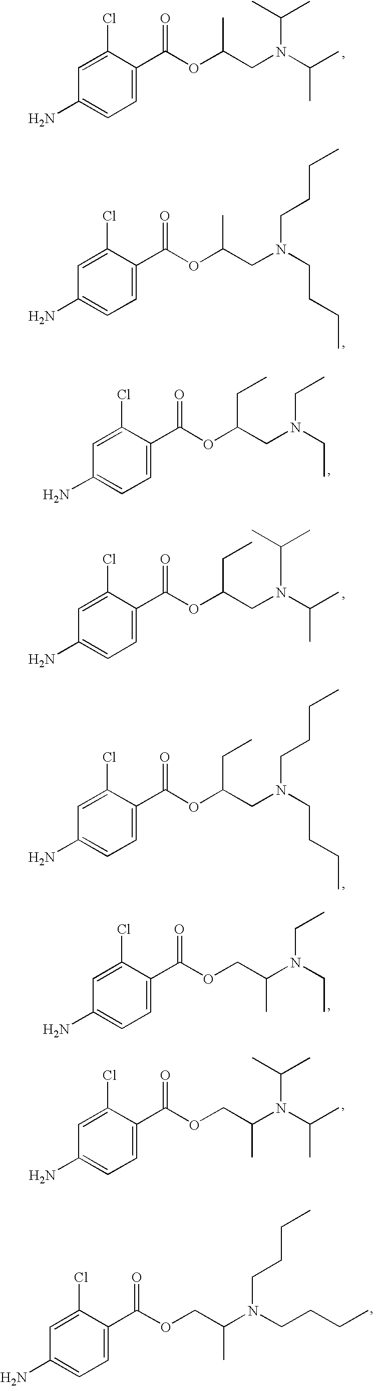

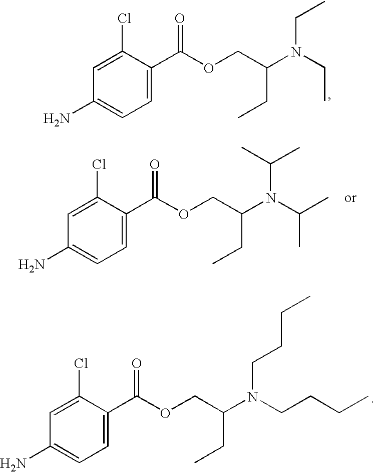

In another embodiment the invention provides a compound of formula II:

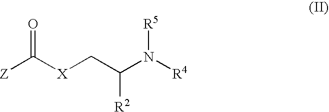

or a prodrug, salt, hydrate, solvate or N-oxide thereof, wherein:

Z is a group of the following formula:

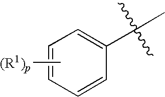

each R¹ is independently hydrogen, —F, —Cl, —Br, —I, —R⁶, —OR⁶, —SR⁶, —NR⁶R⁷ or —C(O)NR⁶R⁷;

p is an integer from 1 to 3;

X is —O— or —S—;

R² is the sidechain of an amino acid;

R⁴ and R⁵ are independently (C1-C6) alkyl optionally substituted with one or more of the same or different R⁸ groups or optionally R⁴ and R⁵ together with the nitrogen atom to which they are bonded form a cycloheteroalkyl ring;

R⁶ and R⁷ are independently hydrogen, alkyl optionally substituted with one or more of the same or different R⁸ groups, cycloalkyl optionally substituted with one or more of the same or different R⁸ groups, cycloheteroalkyl optionally substituted with one or more of the same or different R⁸ groups, aryl optionally substituted with one or more of the same or different R⁸ groups or heteroaryl optionally substituted with one or more of the same or different R⁸ groups; and

R⁸ is —F, —Cl, —Br, —I, —R⁶, —OR⁶, —SR⁶, —NR⁶R⁷, —CF₃, —CN, —C(O)R⁶, —C(O)OR⁶, —C(O)SR⁶, —C(O)NR⁶R⁷, or aryl which is optionally substituted with one or more —F, —Cl, —Br, —I, —R⁶, —OR⁶, —SR⁶, —NR⁶R⁷, —CF₃, —CN, —C(O)R⁶, —C(O)OR⁶, —C(O)SR⁶, or —C(O)NR⁶R⁷.

In another embodiment the invention provides a compound of formula Ia:

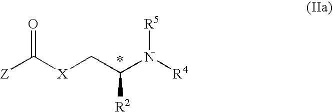

or a prodrug, salt, hydrate, solvate or N-oxide thereof wherein the center marked by * in the formula has the absolute configuration shown.

In another embodiment the invention provides a compound of formula IIb:

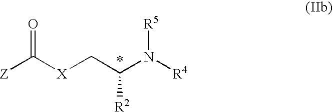

or a prodrug, salt, hydrate, solvate or N-oxide thereof wherein the center marked by * in the formula has the absolute configuration shown.

In another embodiment of the invention Z is:

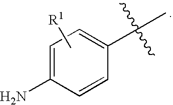

In another embodiment of the invention Z is:

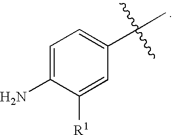

In another embodiment of the invention Z is:

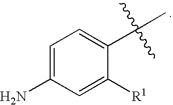

In another embodiment of the invention R¹ is —OR⁶; and R⁶ is alkyl optionally substituted with one or more of the same or different R⁸ groups.

In another embodiment of the invention R¹ is —OR⁶; and R⁶ is (C1-C6)alkyl.

In another embodiment of the invention R¹ is ethoxy, propoxy, or butoxy.

In another embodiment of the invention R² is the sidechain of a natural amino acid.

In another embodiment of the invention R² is methyl, 3-guanidinopropyl, aminocarbonylmethyl, carboxymethyl, mercaptomethyl, 2-carboxy-2-aminoethyldithiomethyl, 2-carboxyethyl, 2-(aminocarbonyl)ethyl, imidazolylmethyl, 4-amino-3-hydroxybutyl, 4-aminobutyl, 2-(methylthio)ethyl, hydroxymethyl, 1-hydroxyethyl, indolylmethyl, 4-hydroxybenzyl, isopropyl, 2-methylpropyl, 1-methylpropyl, or benzyl.

In another embodiment of the invention R² is methyl, isopropyl, 2-methylpropyl, 1-methylpropyl, or benzyl.

In another embodiment of the invention the center marked with * in a compound of formula IIa or IIb has an R absolute configuration.

In another embodiment of the invention the center marked with * in a compound of formula IIa or IIb has an S absolute configuration.

In another embodiment of the invention R⁴ and R⁵ are each independently methyl or ethyl.

In another embodiment of the invention R⁴ is hydrogen.

In another embodiment of the invention R⁴ and R⁵ are each hydrogen.

In another embodiment of the invention R⁴ and R⁵ together with the nitrogen atom to which they are bonded form a piperadino ring.

In another embodiment of the invention X is O.

In another embodiment of the invention n is 2, 3, or 4.

In another embodiment of the invention R² is (C3-C6) alkyl.

In another embodiment of the invention R² propyl, isopropyl, butyl, isobutyl, secbutyl, pentyl, isopentyl, secpentyl, or hexyl.

In another embodiment of the invention the compound of formula I is,

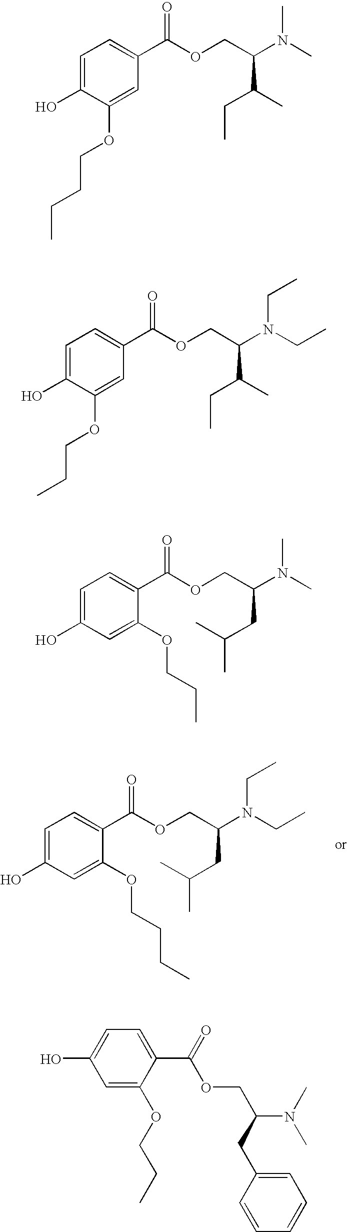

In another embodiment of the invention the compound of formula I is,

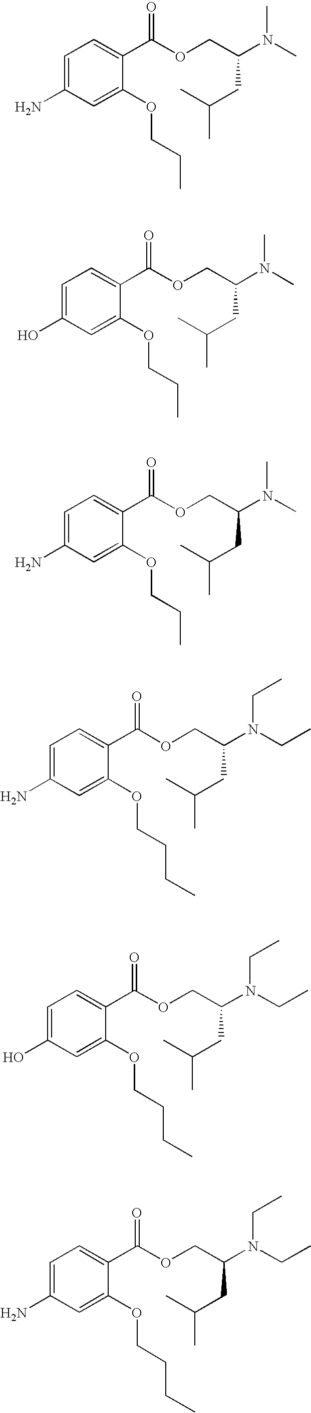

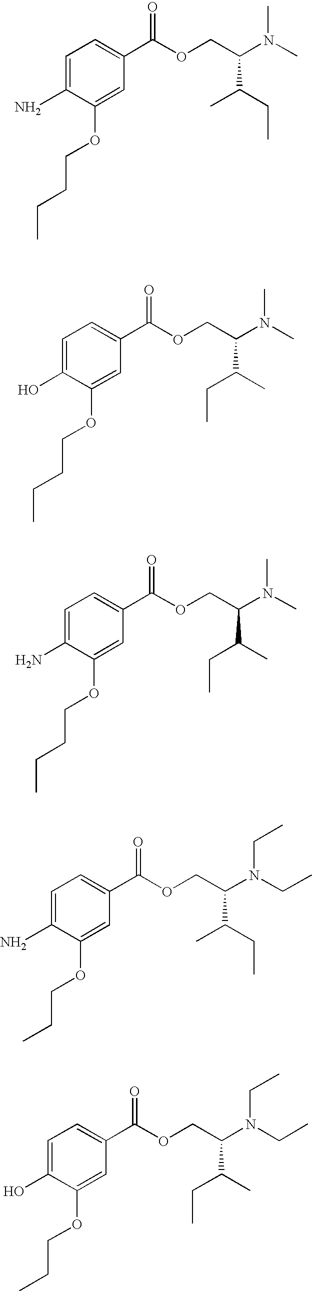

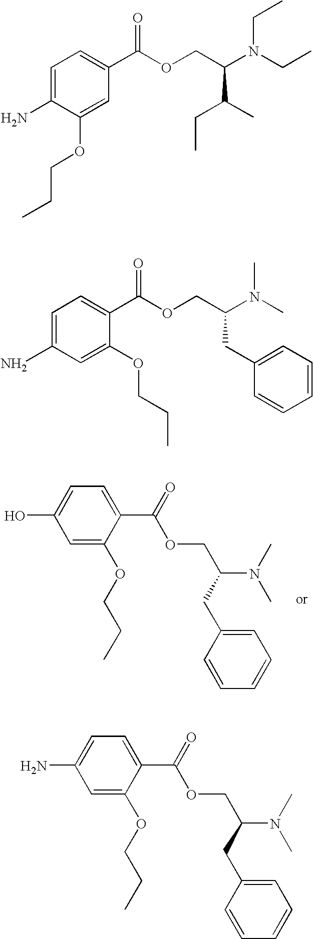

In another embodiment of the invention the compound of formula I is,

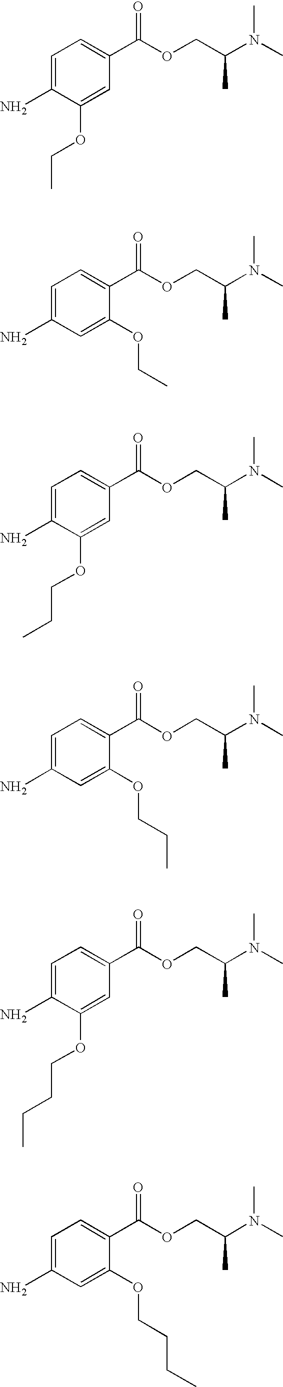

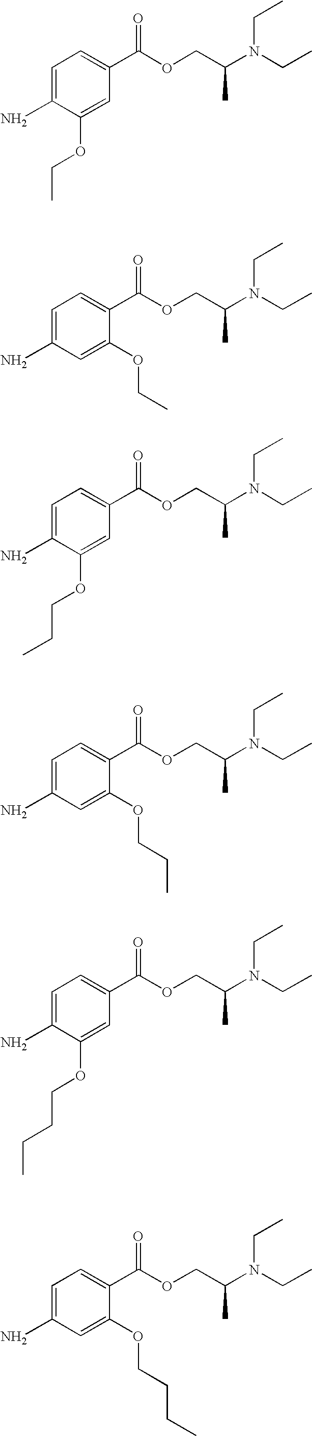

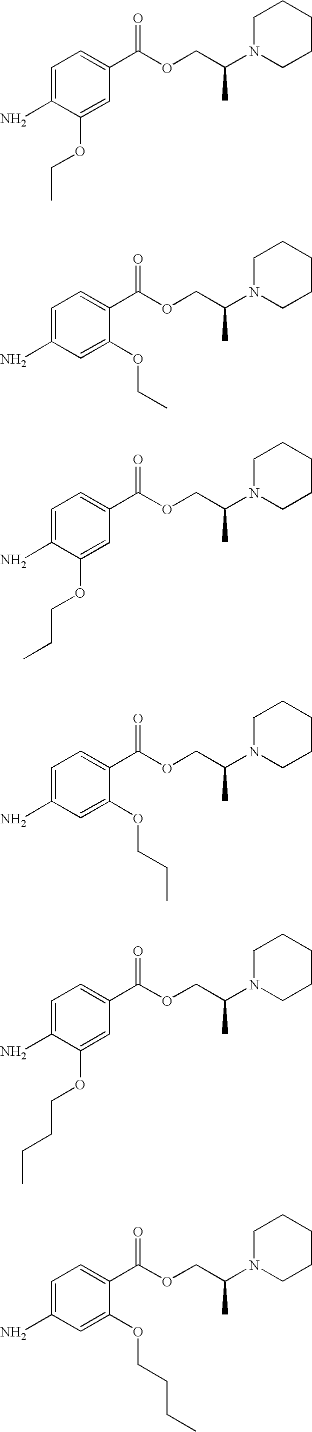

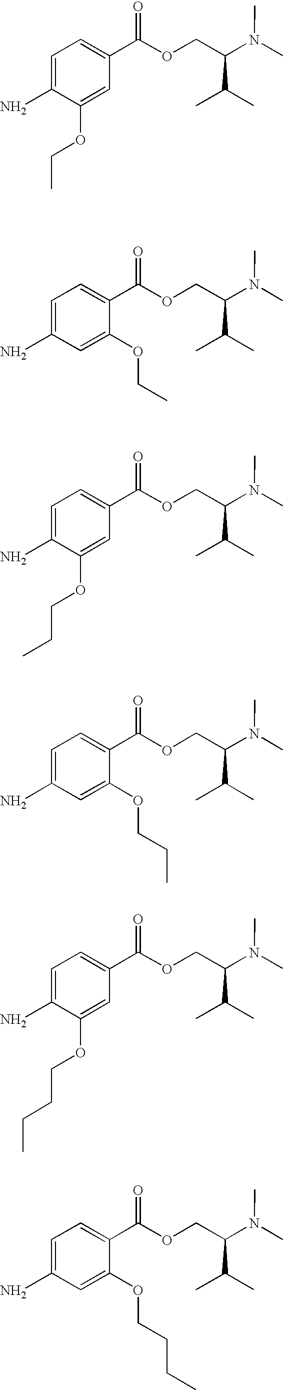

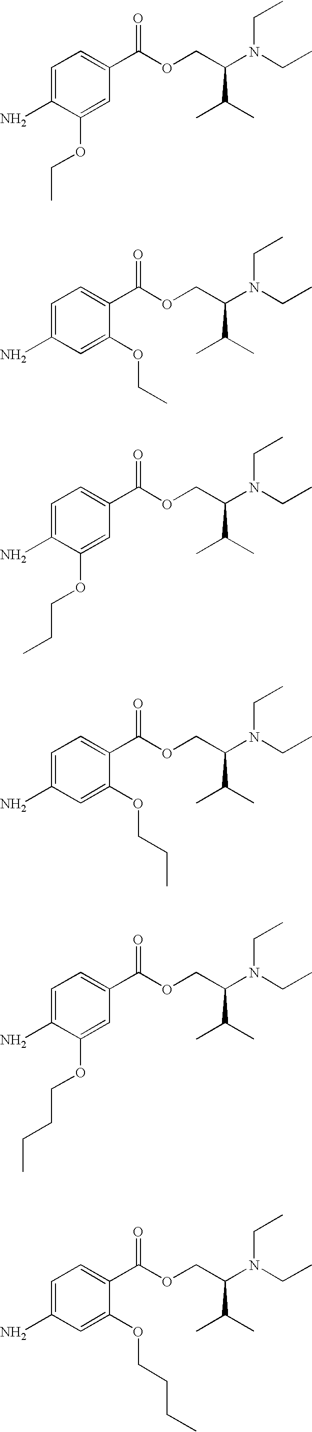

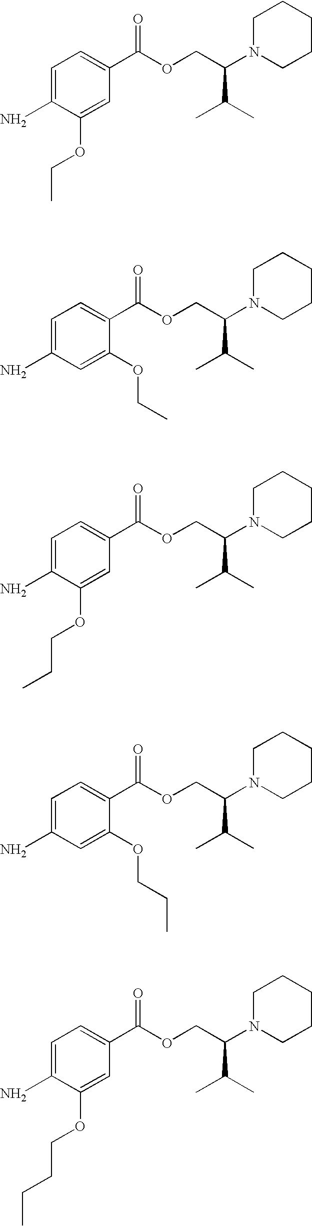

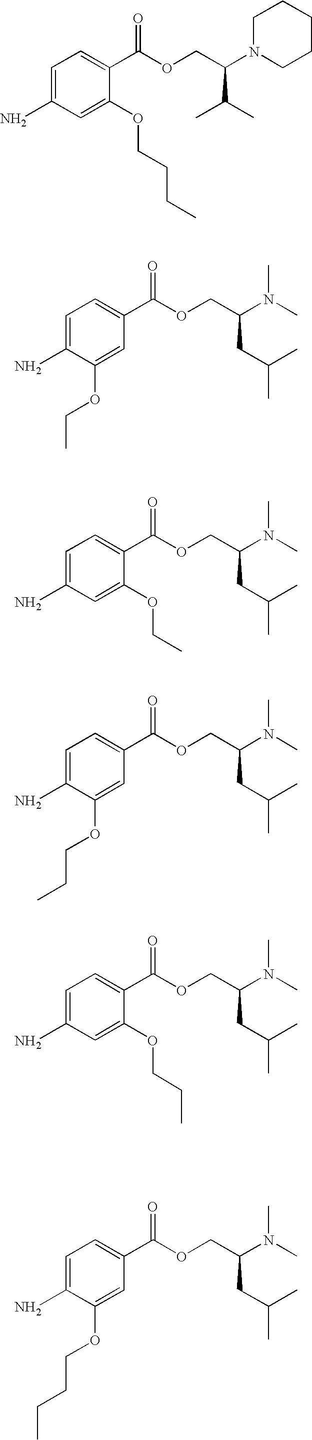

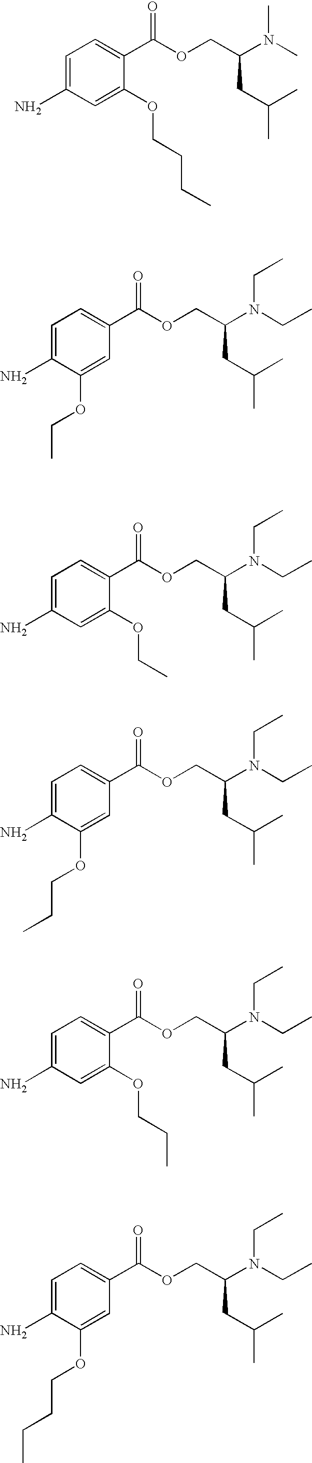

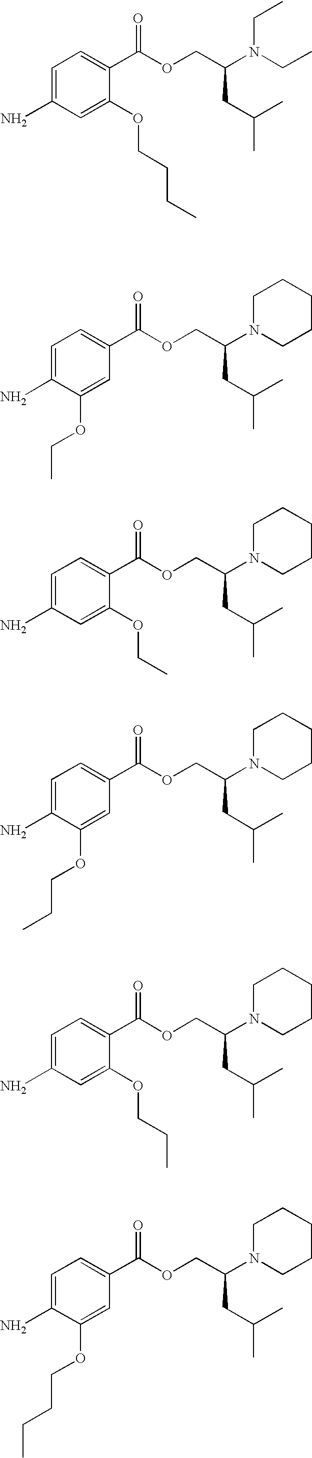

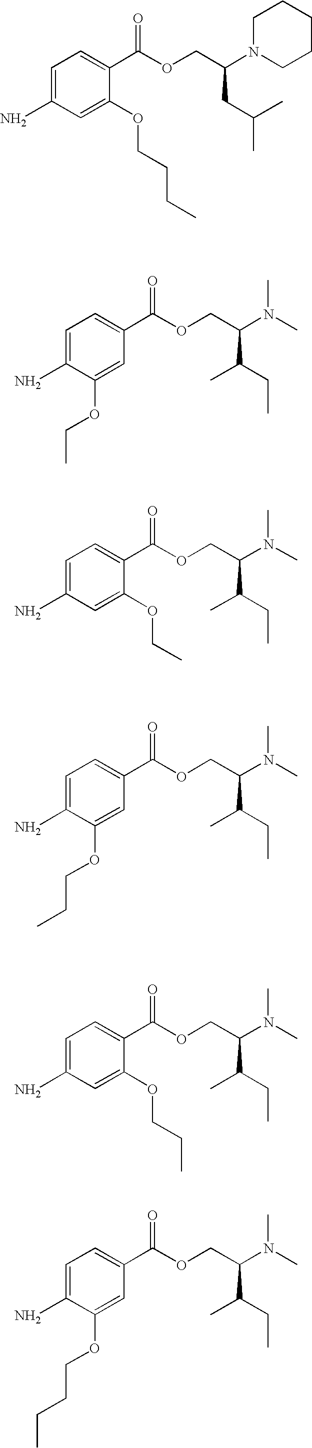

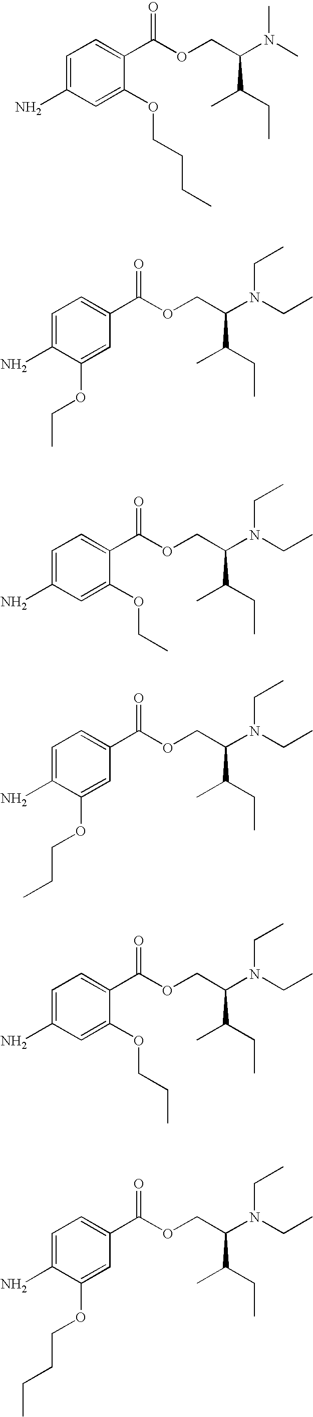

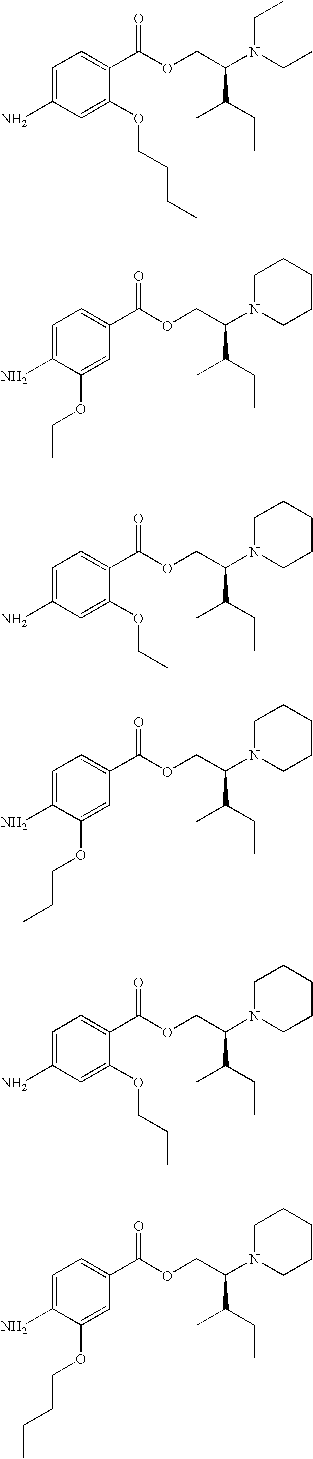

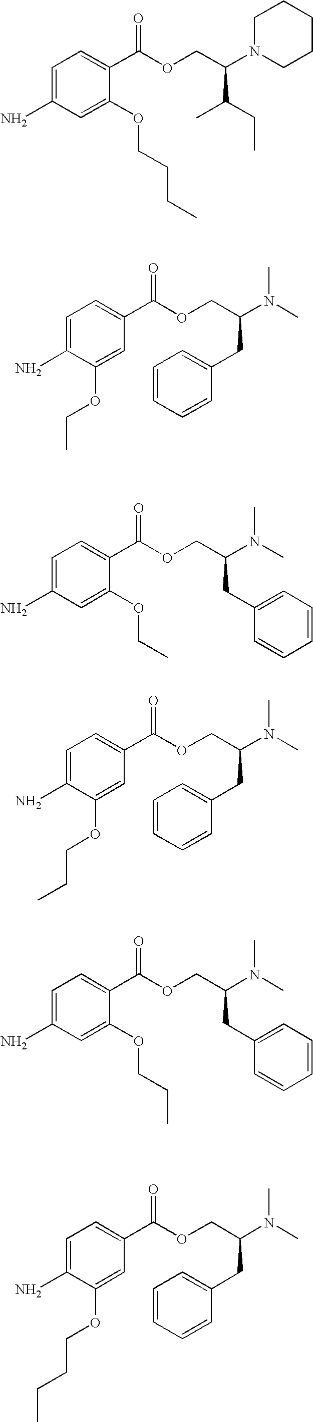

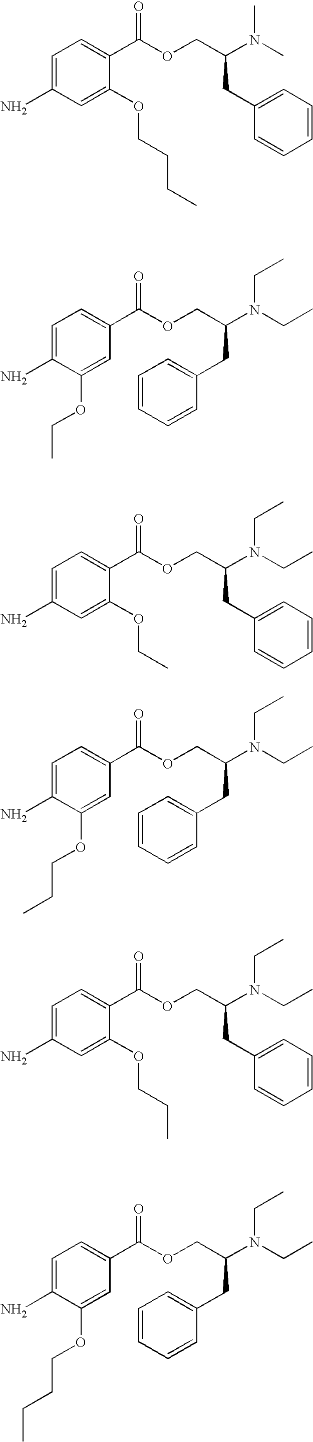

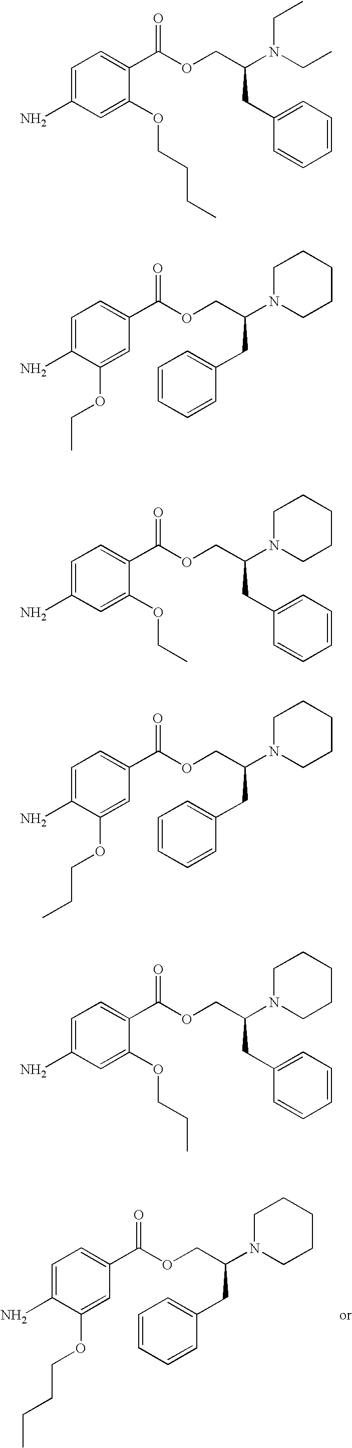

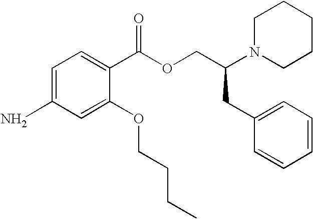

### Transdermal Administration

The transdermal delivery of drugs has become a proven technology that offers a variety of significant clinical benefits over alternative routes of administration. Because transdermal drug delivery offers sustained and controlled release of the drug into the patient, it enables a steady blood-level to be maintained for an extended period of time. This often results in reduced systemic side effects and, sometimes, improved efficacy over other dosage forms.

The efficiency of drug transport into or through the skin depends on a number of factors such as the condition and type of skin, the physicochemical characteristics of the permeant (the drug), other chemicals present in the dosage form (e.g. penetration enhancers), and external conditions (e.g. temperature). The factors with perhaps the greatest influence are the physicochemical characteristics of the drug molecule. Based on the current understanding of the physicochemical features of molecules that are efficiently transported into and through the skin, it can be concluded that many of the compounds of formula I, formula Ia, and formula Ib possess favorable physicochemical characteristics for transdermal penetration. These characteristics include parameters such as LogP (the log of the octonol:water partition coefficient), aqueous solubility of the freebase form, and the molecular weight/molar volume. Accordingly, in one embodiment the invention provides compounds of formula I that possess physicochemical characteristics that are favorable for transdermal penetration.

### Methods of Synthesis

The compounds described herein may typically be obtained via the route illustrated in Scheme 1. The compounds may also be prepared by other procedures known to those of skill in the art (See e.g., Green et al., “Protective Groups in Organic Chemistry,” (Wiley, 2^{nd} ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, (Wiley Interscience); Trost et al., “Comprehensive Organic Synthesis,” (Pergamon Press, 1991); “Theilheimer's Synthetic Methods of Organic Chemistry,” Volumes 1-45, (Karger, 1991); March, “Advanced Organic Chemistry,” (Wiley Interscience), 1991; Larock “Comprehensive Organic Transformations,” (VCH Publishers, 1989); Paquette, “Encyclopedia of Reagents for Organic Synthesis,” (John Wiley & Sons, 1995), Bodanzsky, “Principles of Peptide Synthesis,” (Springer Verlag, 1984); Bodanzsky, “Practice of Peptide Synthesis,” (Springer Verlag, 1984). Further, starting materials may be obtained from commercial sources or via well established synthetic procedures, supra.

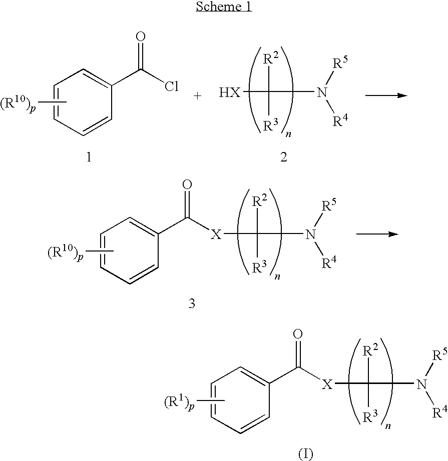

As illustrated in Scheme 1, where each R¹⁰ is independently hydrogen, —F, —Cl, —Br, —I, —R⁶, —OR⁶, —SR⁶, —NO₂ or —C(O)NR⁶R⁷, and X, R², R³, R⁴, R⁵ and n are as previously defined the acid chloride 1 is condensed with amino alcohol or amino thiol 2 to provide compound 3 which can be hydrogenated to provide aryl amine (I) if necessary. Those of skill in the art will appreciate that reduction is only necessary if the compound of Formula (I) is substituted with an amino group on the aromatic ring. Further, as known to the skilled artisan, a carboxylic acid can be condensed with amino alcohol or amino thiol 2 using conventional procedures known in the art. The amino alcohol and/or amino thiol 2 is either commercially available or synthesized from commercially available starting materials using conventional chemistry known to those of skill in the art. Similarly, the benzoyl chloride is available from commercially available precursors using conventional methods.

Selection of appropriate protecting groups, reagents and reaction conditions for any of the steps in the above Scheme is well within the ambit of those of skill in the art. Other methods for synthesis of the compounds described herein will be readily apparent to the skilled artisan and may be used to provide the compounds described herein. Accordingly, the methods presented in the Schemes herein are illustrative rather than comprehensive.

### Therapeutic Methods of Use

In general, the compounds disclosed herein or pharmaceutical compositions thereof may be used to induce and/or maintain local anesthesia and analgesia and are particularly useful for the prophylaxis and/or treatment of pain. As local anesthetics, the compounds disclosed herein or pharmaceutical compositions thereof are useful for regional anesthesia, e.g., topical anesthesia, infiltration anesthesia, perisurgical tissue anesthesia, field block anesthesia, peripheral nerve block anesthesia, epidural anesthesia, spinal anesthesia, bier block anesthesia (local anesthetic injection into an extremity isolated by a tourniquet) and combinations thereof. The compounds disclosed herein or pharmaceutical compositions thereof may also be used to relieve or prevent the pain associated with venipuncture, lumbar puncture, myringtomy, arterial cannulation, neuropathic pain, trauma and tissue ischemia. The compounds disclosed herein or pharmaceutical compositions thereof may be applied topically via patches, or other reservoir systems, bandages or gauzes, creams, ointments or other transdermal delivery systems to treat and/or prevent the pain associated with, for examples, dermatoses, hemorrhoids and burns.

### Pharmaceutical Compositions

The pharmaceutical compositions disclosed herein comprise a local anesthetic disclosed herein with a suitable amount of a pharmaceutically acceptable vehicle, so as to provide a form for proper administration to a subject.

Suitable pharmaceutical vehicles include excipients such as starch, glucose, lactose, sucrose, gelatin, malt, rice, flour, chalk, silica gel, sodium stearate, glycerol monostearate, talc, sodium chloride, cellulose, hydroxycellulose, lactose, methylcellulose, polyvinylpyrrolidone, microcrystalline cellulose, gum acacia, dried skim milk, glycerol, propylene, glycol, water, ethanol, polyoxyethylene sorbitan derivatives and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in any conventional manner using one or more physiologically acceptable carriers, diluents, excipients or auxiliaries, which facilitate processing of compositions and compounds disclosed herein into preparations which can be used pharmaceutically. Proper formulation is dependent upon the route of administration chosen.

The present pharmaceutical compositions can take the form of solutions, suspensions, emulsions, powders, sustained-release formulations, aerosols, sprays, suspensions or any other form suitable for use known to the skilled artisan. Other examples of suitable pharmaceutical vehicles have been described in the art (see Remington's Pharmaceutical Sciences, Philadelphia College of Pharmacy and Science, 19th Edition, 1995).

In still other embodiments, the dosage form comprises compounds disclosed herein coated on a polymer substrate. The polymer can be an erodible, or a nonerodible polymer. The coated substrate may be folded onto itself to provide a bilayer polymer drug dosage form. For example, compounds disclosed herein can be coated onto a polymer such as a polypeptide, collagen, gelatin, polyvinyl alcohol, polyorthoester, polyacetyl, or a polyorthocarbonate and the coated polymer folded onto itself to provide a bilaminated dosage form. In operation, the bioerodible dosage form erodes at a controlled rate to dispense the compounds over a sustained release period. Representative biodegradable polymers comprise a member selected from the group consisting of biodegradable poly(amides), poly(amino acids), poly(esters), poly(lactic acid), poly(glycolic acid), poly(carbohydrate), poly(orthoester), poly (orthocarbonate), poly(acetyl), poly(anhydrides), biodegradable poly(dihydropyrans), and poly(dioxinones) which are known in the art (Rosoff, Controlled Release of Drugs, Chap. 2, pp. 53-95 (1989); Heller et al., U.S. Pat. No. 3,811,444; Michaels, U.S. Pat. No. 3,962,414; Capozza, U.S. Pat. No. 4,066,747; Schmitt, U.S. Pat. No. 4,070,347; Choi et al., U.S. Pat. No. 4,079,038; Choi et al., U.S. Pat. No. 4,093,709).

In other embodiments, the dosage form comprises compounds disclosed herein loaded into a polymer that releases the drug(s) by diffusion through a polymer, or by flux through pores or by rupture of a polymer matrix. The drug delivery polymeric dosage form comprises a concentration of 10 mg to 2500 mg homogenously contained in or on a polymer. The dosage form comprises at least one exposed surface at the beginning of dose delivery. The non-exposed surface, when present, is coated with a pharmaceutically acceptable material impermeable to the passage of the drug(s). The dosage form may be manufactured by procedures known in the art. An example of providing a dosage form comprises blending a pharmaceutically acceptable carrier like polyethylene glycol, with a known dose of compositions and/or compounds disclosed herein at an elevated temperature, (e.g., 37° C.), and adding it to a silastic medical grade elastomer with a cross-linking agent, for example, octanoate, followed by casting in a mold. The step is repeated for each optional successive layer. The system is allowed to set for about 1 hour, to provide the dosage form. Representative polymers for manufacturing the dosage form comprise a member selected from the group consisting of olefin, and vinyl polymers, addition polymers, condensation polymers, carbohydrate polymers, and silicone polymers as represented by polyethylene, polypropylene, polyvinyl acetate, polymethylacrylate, polyisobutylmethacrylate, poly alginate, polyamide and polysilicone. The polymers and procedures for manufacturing them have been described in the art (Coleman et al., Polymers 1990, 31, 1187-1231; Roerdink et al., Drug Carrier Systems 1989, 9, 57-10; Leong et al., Adv. Drug Delivery Rev. 1987, 1, 199-233; Roff et al., Handbook of Common Polymers 1971, CRC Press; Chien et al., U.S. Pat. No. 3,992,518).

For topical administration a compound disclosed herein may be formulated as emulsions, solutions, gels, ointments, creams, suspensions, jellies etc. as are well-known in the art.

Systemic formulations include those designed for administration by injection, e.g., subcutaneous, intravenous, intramuscular, intrathecal, infiltration or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include but are not limited to sodium channel blockers, antibiotics, N-acetyl cysteine, homocysteine and phospholipids.

For injection, compounds disclosed herein may be formulated in aqueous solutions, such as physiologically compatible buffers such as Hanks' solution, Ringer's solution, physiological saline buffer or in the form of an emulsion (as a water-in-oil or oil-in-water emulsion). In one embodiment of the invention, the compound is formulated for injection with an organic acid buffer system (e.g. a (C1-C6) organic acid such as citric, succinic, or acetic acid). The solution may also contain formulatory agents such as suspending, stabilizing and/or dispersing agents. Alternatively, compounds disclosed herein may be in powder form for constitution with a suitable vehicle, e.g., sterile pyrogen-free water, before use.

Vasoconstrictors (e.g., epinephrine or phenylepinephrine), corticosteroids (demaxthasone, cortisone, hydrocortisone, prednisone, beclamethasone, betamethasone, flunisolide, methylprednisone, prednisolone, triamcinolone, alcolmetasone, amcinonide, clobestal, fludrocortisone, difluorsone diacetate, fluocinolone acetonide, fluoromethalone, flurandrenolide, halcinonide, medrysone, etc.) and/or permeability enhancers (e.g., sodium cholate, sodium glycocholate, sodium glycodeoxycholate, taurodeoxycholate, sodium deoxycholate, sodium lithiocholate, chenocholate, chenodeoxycholate, ursocholate, ursodeoxycholate, hydrodeoxycholate, dehydrocholate, glycochenolate, taurochenocholate, taurochenodeoxycholate, etc.) can also be added to the present pharmaceutical compositions.

### Therapeutic/Prophylactic Administration and Doses

When used to maintain and/or induce anesthesia and/or analgesia, the compounds disclosed herein and/or pharmaceutical compositions thereof may be administered alone or in combination with other pharmaceutical agents including compounds disclosed herein and/or pharmaceutical compositions thereof. The compounds disclosed herein may be administered or applied per se or as pharmaceutical compositions. The specific pharmaceutical composition depends on the desired mode of administration, as is well known to the skilled artisan.

Compounds disclosed herein and/or pharmaceutical compositions thereof may be administered to a subject by injection including intravenous injection, continuous infusion, intramuscular injection, subcutaneous injection, transdermally, intracerebrally, intravaginally, rectally, topically, particularly to the ears, nose, eyes, or skin or any other convenient method known to those of skill in the art. In some embodiments, compounds disclosed herein and/or pharmaceutical compositions thereof are delivered by infiltration methods such as, for example, peripheral nerve blocks, serosal and neuraxial delivery, (e.g., epidural, caudal, etc.)

Transdermal devices can also be used to deliver the compounds disclosed herein and/or pharmaceutical compositions thereof. In some embodiments, the transdermal device is a matrix type transdermal device (Miller et al., International Publication No. WO 2004/041324). In other embodiments, the transdermal device is a multi-laminate transdermal device (Miller, United States Patent Application Publication No. 2005/0037059).

The amount of compounds disclosed herein and/or pharmaceutical compositions thereof that will be effective in the treatment or prevention of pain in a patient will depend on the specific nature of the pain condition and can be determined by standard clinical techniques known in the art. The amount of compounds disclosed herein and/or pharmaceutical compositions thereof administered will, of course, be dependent on, among other factors, the subject being treated, the weight of the subject, the severity of the affliction, the manner of administration and the judgment of the prescribing physician.

### Combination Therapy

In certain embodiments, compounds disclosed herein and/or pharmaceutical compositions thereof can be used in combination therapy with other therapeutic agents, such as, for example, other local ester anesthetics and/or local amide anesthetics. The compounds disclosed herein and/or pharmaceutical compositions thereof and the therapeutic agent can act additively or, more preferably, synergistically. In some embodiments, compounds disclosed herein and/or pharmaceutical compositions thereof are administered concurrently with the administration of another therapeutic agent. For example, compounds disclosed herein and/or pharmaceutical compositions thereof may be administered together with another therapeutic agent. In other embodiments, compounds disclosed herein and/or pharmaceutical compositions thereof are administered prior or subsequent to administration of other therapeutic agents.

### Screening

The quality and duration of local anesthetic block produced by a test compound can be evaluated using the following Rat Sciatic Nerve Assay.

### Rat Sciatic Nerve Assay

This assay enables evaluation of the efficacy of local anesthetic molecules via application of the compounds adjacent to the sciatic nerve in the rat and measuring the anesthetic effects on response to pain stimulus (toe pinch), motor function (postural hindlimb thrust) and proprioception (ability of the animal to balance) measured over time.

Each rat was tested for baseline hindpaw motor, proprioceptive, and sensory function and then injected with a test solution over the right sciatic nerve. All tests were performed bilaterally starting on the side that was not injected.

Sciatic nerve injection: Rats were shaved over the right sciatic area. Rats were lightly anesthetized with 1.5% isoflurane until it was possible to manipulate the hip joint. Test Solution(s) (200 uL) were injected proximal to the sciatic nerve using an insulin syringe directed between the greater trochanter and the ischial tuberosity.

Evaluations of anesthetic block included motor, sensory, and proprioceptive block assessed from injection time until all forms of block are absent.

Motor function was evaluated by measuring the extensor postural thrust of the bilateral hind limbs. The rat was held upright over a scale with the hind limbs extended so that the body weight is supported by the distal metatarsus and toes of one hindlimb. The extensor thrust was measured as the gram force applied to the scale as the rat is pushed down until the heel touches the balance. The pretreatment control value was considered to be 0% of the maximal possible effect (MPE). The reduction in this force, representing reduced extensor muscle contraction induced by motor blockade, was calculated as percentage of the control force. A force <20 g was considered to be 100% of the MPE.

Proprioception was evaluated with a balancing test. A hopping response was evoked by lifting the rat into a vertical position with hindlimbs resting on the table and lifting one hindlimb at a time off the table top so that the animal's weight was resting on one hindlimb, then moving the rat laterally until it hopped to the side to remain upright. A predominantly motor impairment caused a prompt but weaker than normal response. Conversely, with a predominantly proprioceptive blockade, delayed hopping was followed by greater lateral hops to avoid falling over or, in case of full blockade, no hopping at all. The proprioceptive deficit was compared to the baseline response and graded as 4 (equal to baseline or 0% MPE), 3 (slightly impaired), 2 (moderately impaired), 1 (severely impaired) and 0 (complete or 100% MPE).

Nociceptive responses were measured with a forceps pinch test. The withdrawal response to forceps pinch applied to the distal phalanx of digit 5 was compared to the baseline response and graded as 4 (equal to baseline or 0% MPE), 3 (slightly impaired), 2 (moderately impaired), 1 (severely impaired), and 0 (absent or 100% MPE).

Representative compounds of the invention that were tested in the above Rat Sciatic Nerve Assay were found to demonstrate clinically relevant local anesthetic activity. In all compounds tested, the efficacy (time of complete conduction block) was superior to that demonstrated by lidocaine. In most cases, the efficacy was superior to that demonstrated by bupivacaine. Full clinical recovery was observed in all animals tested, demonstrating that the nerve blocks are fully reversible (i.e. the conduction block is likely not due to nerve damage induced by the compounds).

The invention will now be illustrated by the following non-limiting Examples.

### EXAMPLES

### Example 1

### Synthesis of Compound 1

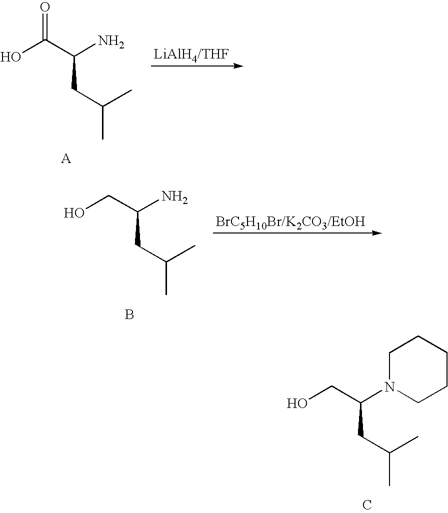

a. Synthesis of Intermediate B:

To a solution of dry THF (300 ml) was slowly charged lithium aluminum hydride (10.84 g, 0.151 mol) at 0° C. over 30 minutes. To this reaction mixture was charged, in portions, L-leucine (25.0 g, 0.213 mol) over a period of 45 minutes while maintaining the reaction mixture temperature between 0-5° C. to control the vigorous evolution of hydrogen. The resulting reaction mixture was then heated to reflux and maintained at reflux for 16 hours at which time it was cooled to 0-5° C., diluted with diethyl ether (300 ml) and slowly quenched with DI water (12 ml). To the quenched solution was charged 15% w/v NaOH solution (12 ml) which resulted in the precipitation of a white solid. The slurry was stirred at room temperature for 30 minutes and the white solid was removed by filtration to give a clear organic filtrate which was dried with sodium sulfate (q.s.). The sodium sulfate was removed by filtration and the organic solution was concentrated under reduced pressure to afford B as a yellow liquid (18 g, 82%).

b. Synthesis of Intermediate C:

To a solution of absolute ethanol (200 ml) was charged B (10 g, 0.08 mol) followed by dried potassium carbonate (58.9 g, 0.42 mol) and to the resulting slurry was charged, in drop wise fashion, 1,5-dibromopentane (23.35 ml, 0.17 mol). The reaction mixture was then heated to reflux and maintained at reflux for 48 hours, then cooled to room temperature and filtered through a celite bed. The organic layer was concentrated under reduced pressure to give an oil which was purified via silica gel chromatography, using petroleum ether:ethyl acetate as eluent. The fractions containing Compound C were combined and the organic was evaporated under reduced pressure to give Compound C as a yellow liquid (8.9 g, 56%).

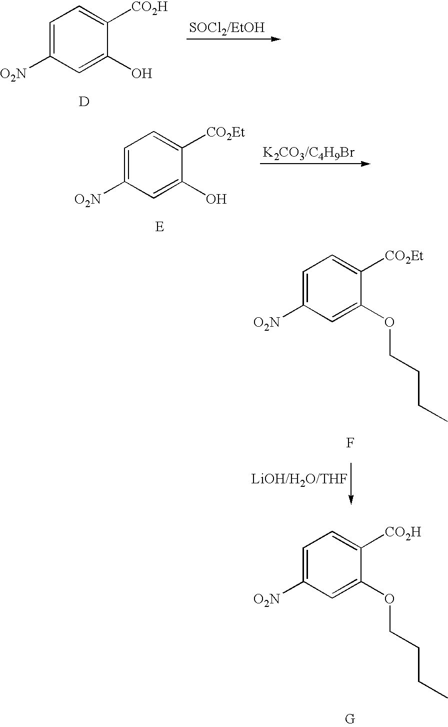

c. Synthesis of Intermediate E:

To a solution of anhydrous ethanol (500 ml) was charged D (50 g, 0.27 mol) and the reaction mixture was cooled to 0-5° C. at which time thionyl chloride (59.6 ml, 0.81 mol) was slowly added to keep the reaction mixture temperature <20° C. After complete thionyl chloride addition the reaction mixture was heated to reflux and maintained at reflux overnight. The reaction mixture was cooled to 20° C. and then concentrated to an oil by evaporation under reduced pressure. To the residue was charged a mixture of water (200 ml) and ethyl acetate (200 ml). The layers were separated and the aqueous was extracted with additional ethyl acetate (2×200 ml). The combined organic layers were washed with 10% sodium bicarbonate solution (200 ml), water (200 ml) and brine (200 ml) and then dried over sodium sulfate, filtered and concentrated under reduced pressure to give E as a yellow solid (51 g, 89%).

d. Synthesis of Intermediate F:

To a solution of DMF (250 ml) was charged E (25 g, 0.11 mol) followed by potassium carbonate (49.08 g, 0.35 mol) and the resulting slurry was cooled to 0-5° C. at which time n-butylbromide (16.6 ml, 0.15 mol) was charged and the reaction mixture was stirred at room temperature for 24 hours. To the reaction mixture was charged ethyl acetate (250 ml) and the slurry was filtered through a bed of celite. The organic filtrate was washed with water (3×200 ml) and brine (200 ml) and then dried with sodium sulfate (q.s.) and concentrated under reduced pressure to give F as a yellow solid (28 g, 88%).

e. Synthesis of Intermediate G:

To a solution of THF (150 ml) and water (150 ml) was charged F (14.98 g, 0.062 mol) and to this reaction mixture was charged a solution of lithium hydroxide (7.5 g, 0.31 mol) in 50 ml of water, and the resulting mixture was stirred at room temperature overnight. The reaction mixture was concentrated under reduced pressure to ⅓ its original volume and 1.5 N HCl solution was added until the pH of the solution was <6. The reaction mixture was extracted with ethyl acetate (2×200 ml) and the combined organic layer was washed with water (100 ml) and then brine (100 ml), then dried with sodium sulfate (q.s.), concentrated under reduced pressure to give Compound G as a yellow solid (13 g, 97%).

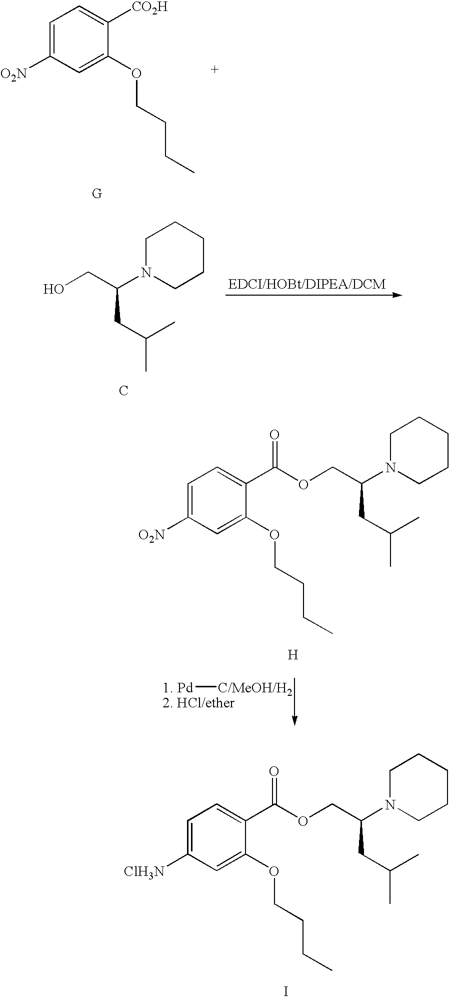

f. Synthesis of Intermediate H:

To a solution of dichloromethane (30 ml) was charged Compound G (2.84 g, 0.01 mol), C (2 g, 0.011 mol), Hunig's base (5.5 ml, 0.032 mol), EDCI (1.94 g, 0.016 mol) and HOBt (0.36 g, 0.0027 mol) and the reaction mixture was stirred at room temperature overnight. The reaction mixture was then diluted with water (50 ml) and extracted with dichloromethane (100 ml). The combined organic layers were washed with 10% sodium bicarbonate solution (50 ml), water (50 ml) and then brine (50 ml). The solution was then dried with sodium sulfate (q.s.), filtered and concentrated under reduced pressure to give a brown liquid which was purified by silica gel chromatography using 5% ethyl acetate/95% petroleum ether as eluent. The combined fractions were concentrated under reduced pressure to give Compound H as a yellow liquid (2 g, 46%).

g. Synthesis of Compound 1 Hydrochloride Salt:

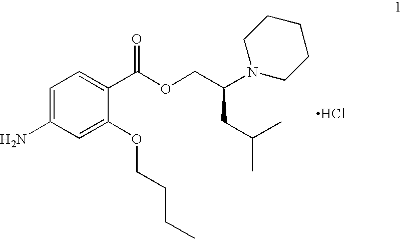

To a solution of methanol (50 ml) was charged Compound H (2 g, 0.004 mol) followed by Pd—C (0.2 g, 10 mol %) and hydrogen gas was introduced and the reaction mixture was hydrogenated at 3.5 kg/cm2 for 2 hours. The reaction mixture was then filtered over celite and the filtrate was concentrated under reduced pressure and the residue was purified using silica gel chromatography eluting with 1.5% methanol in chloroform. The combined fractions were evaporated under reduced pressure to give Compound 1 free base as a brown liquid (0.238 g, 12%).

To a solution of chloroform (25 ml) was charged Compound 1 free base (0.238 g) and to this solution was charged HCl in diethylether (5 ml) and the reaction mixture was stirred at room temperature for 30 minutes. The solution was concentrated to dryness and diethyl ether was re-charged and the resulting solid was collected and washed with dichloromethane and ethyl acetate to yield the hydrochloride salt of Compound 1 as an off-white solid. HPLC purity (AUC): 95.0%, M+H+=377; 1H NMR (DMSO): δ (ppm)=0.9, m (9H); 1.4, m (3H); 1.7, m (8H); 2.0, m (2H); 3.1, m (2H); 3.3, t (2H); 3.5, bs (1H); 4.0 t (2H); 4.5, m (2H); 6.3, d (1H); 6.4, s (1H); 7.7, d (1H); 10.8, bs (1H).

### Example 2

### Synthesis of Compound 2 Hydrochloride Salt

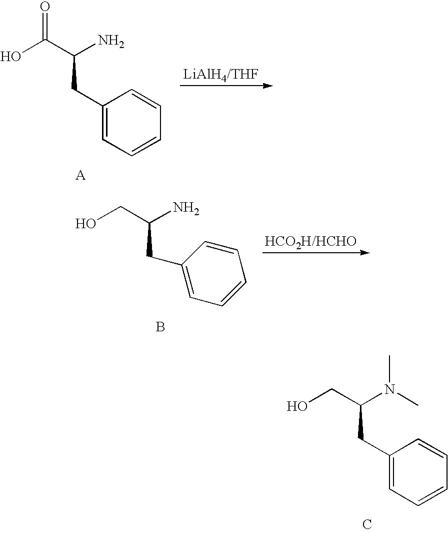

a. Synthesis of Intermediate B:

To a solution of dry THF (300 ml) was slowly charged lithium aluminum hydride (10.84 g, 0.151 mol) at 0° C. over 30 minutes. To this reaction mixture was charged, in portions, L-phenylalanine (25.0 g, 0.227 mol) over a period of 45 minutes while maintaining the reaction mixture temperature between 0-5° C. to control the vigorous evolution of hydrogen. The resulting reaction mixture was then heated to reflux and maintained at reflux for 16 hours at which time it was cooled to 0-5° C., diluted with diethyl ether (300 ml) and slowly quenched with DI water (12 ml). To the quenched solution was charged 15% w/v NaOH solution (12 ml) which resulted in the precipitation of a white solid. The slurry was stirred at room temperature for 30 minutes and the white solid was removed by filtration to give a clear organic filtrate which was dried with sodium sulfate (q.s.). The sodium sulfate was removed by filtration and the organic solution was concentrated under reduced pressure to afford intermediate B as yellow liquid (19 g, 84%).

b. Synthesis of Intermediate C:

To a solution of formic acid (7.48 ml, 0.19 mol) and formaldehyde (5.5 ml, 0.19 mol) was charged intermediate B (10 g, 0.066 mol) and the reaction mixture was heated to reflux at 105° C. for 16 hours. The reaction mixture was then cooled to room temperature and to the cooled solution was charged 10% sodium hydroxide solution followed by ethyl acetate (300 ml). The layers were separated and the organic layer was washed with brine (100 ml), then dried with sodium sulfate, filtered and the filtrate concentrated in vacuo to give intermediate C as a brown liquid (8 g, 67%).

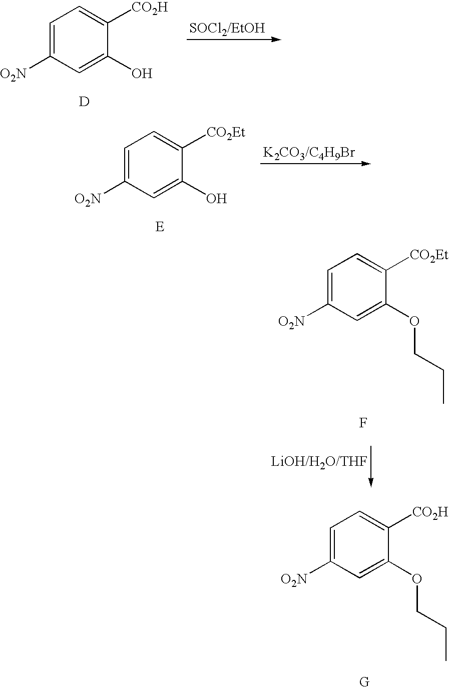

c. Synthesis of Intermediate E:

To a solution of anhydrous ethanol (250 ml) was charged intermediate D (25 g, 0.137 mol) and the reaction mixture was cooled to 0-5 C at which time thionyl chloride (29.8 ml, 0.41 mol) was slowly added to keep the reaction mixture temperature <20 C. After complete thionyl chloride addition the reaction mixture was heated to reflux and maintained at reflux overnight. The reaction mixture was cooled to 20 C and then concentrated to an oil by evaporation under reduced pressure. To the residue was charged a mixture of water (100 ml) and ethyl acetate (100 ml). The layers were separated and the aqueous was extracted with additional ethyl acetate (2×100 ml). The combined organic layers were washed with 10% sodium bicarbonate solution (100 ml), water (100 ml) and brine (100 ml) and then dried over sodium sulfate, filtered and concentrated under reduced pressure to give intermediate E as a yellow solid (27 g, 93%).

d. Synthesis of Intermediate F:

To a solution of DMF (250 ml) was charged intermediate E (27 g, 0.127 mol) followed by potassium carbonate (53 g, 0.383 mol) and the reaction mixture was cooled to 0 C at which time n-propylbromide (12.8 ml, 0.140 mol) was charged and the resulting slurry was stirred at room temperature for 24 hours. The reaction mixture was then diluted with ethyl acetate (250 ml), filtered through a bed of celite and the cake was washed with ethyl acetate (100 ml). The combined organic layers were washed with water (3×200 ml) then brine (200 ml) and the organic layer was dried with sodium sulfate and concentrated in vacuo to give intermediate F as a yellow solid (25 g, 76%).

e. Synthesis of Intermediate G:

To a solution of THF (150 ml) and water (150 ml) was charged intermediate F (21.0 g, 0.083 mol) and to this reaction mixture was charged a solution of lithium hydroxide (10.0 g, 0.418 mol) in 50 ml of water, and the resulting mixture was stirred at room temperature overnight. The reaction mixture was concentrated under reduced pressure to ⅓ its original volume and 1.5 N HCl solution was added until the pH of the solution was <6. The reaction mixture was extracted with ethyl acetate (2×200 ml) and the combined organic layer was washed with water (100 ml) and then brine (100 ml), then dried with sodium sulfate (q.s.), concentrated under reduced pressure to give intermediate G as a yellow solid (20 g, 83%).

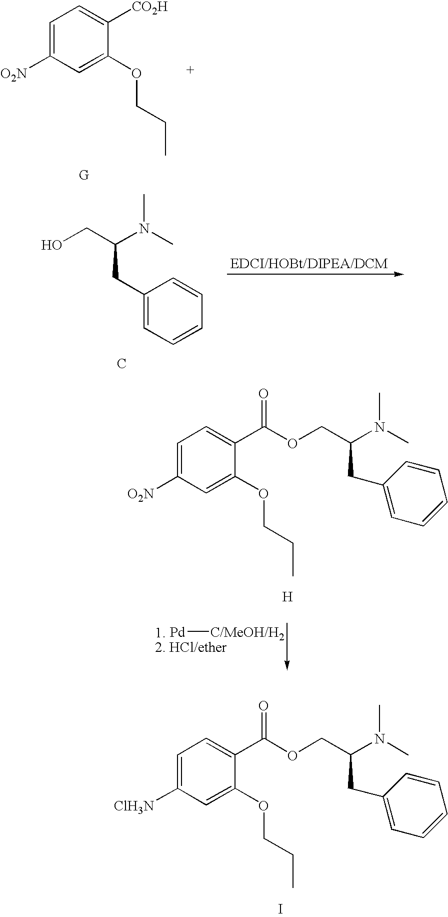

f. Synthesis of Intermediate H:

To a solution of dichloromethane (30 ml) was charged intermediate G (2.76 g, 0.12 mol), C (2 g, 0.011 mol), Hunig's base (5.5 ml, 0.032 mol), EDCI (1.94 g, 0.016 mol) and HOBt (0.36 g, 0.0027 mol) and the reaction mixture was stirred at room temperature overnight. The reaction mixture was then diluted with water (50 ml) and extracted with dichloromethane (100 ml). The combined organic layers were washed with 10% sodium bicarbonate solution (50 ml), water (50 ml) and then brine (50 ml). The solution was then dried with sodium sulfate (q.s.), filtered and concentrated under reduced pressure to give a brown liquid which was purified by silica gel chromatography using 5% ethyl acetate/95% petroleum ether as eluent. The combined fractions were concentrated under reduced pressure to give intermediate H as a yellow liquid (1.3 g, 30%).

g. Synthesis of Compound 2 Hydrochloride Salt (I):

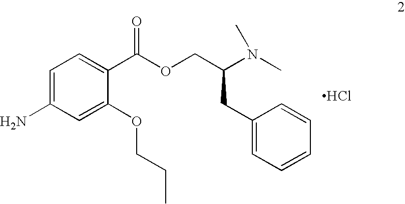

To a solution of methanol (50 ml) was charged intermediate H (1.3 g, 0.0034 mol) followed by Pd—C (0.13 g, 10 mol %) and hydrogen gas was introduced and the reaction mixture was hydrogenated at 3.5 kg/cm2 for 2 hours. The reaction mixture was then filtered over celite and the filtrate was concentrated under reduced pressure and the residue was purified using silica gel chromatography eluting with 1.5% methanol in chloroform. The combined fractions were evaporated under reduced pressure to give Compound 2 free base as a brown liquid (0.44 g, 34%).

To a solution of chloroform (25 ml) was charged Compound 2 free base (0.44 g) and to this solution was charged HCl in diethylether (5 ml) and the reaction mixture was stirred at room temperature for 30 minutes. The solution was concentrated to dryness and diethyl ether was re-charged and the resulting solid was collected and washed with dichloromethane and ethyl acetate to yield the hydrochloride salt of Compound 2 as an off-white solid. HPLC purity (AUC): 98.5%; M+H+=357; 1H NMR (DMSO): δ (ppm)=1.0, t (3H); 1.9, m (2H); 2.5, s (6H); 2.8, m (1H); 3.0, m (1H); 3.1, m (1H); 4.0, t (2H); 4.4, m (2H); 6.8, d (1H); 7.2, m (5H); 7.4, s (1H); 7.5, s (1H).

### Example 3

### Synthesis of Compound 3 Hydrochloride Salt

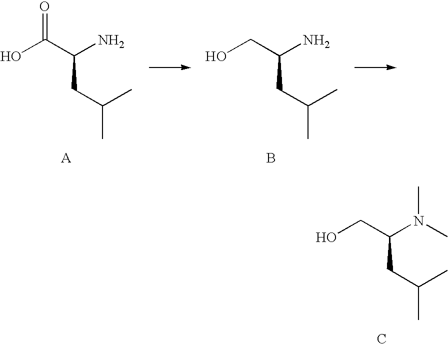

a. Synthesis of Intermediate B:

To a solution of dry THF (250 ml) was slowly charged lithium aluminum hydride (10.84 g, 0.151 mol) at 0 C over 30 minutes. To this reaction mixture was charged, in portions, L-leucine (25.0 g, 0.227 mol) over a period of 45 minutes while maintaining the reaction mixture temperature between 0-5 C to control the vigorous evolution of hydrogen. The resulting reaction mixture was then heated to reflux and maintained at reflux for 16 hours at which time it was cooled to 0-5 C, diluted with diethyl ether (300 ml) and slowly quenched with DI water (12 ml). To the quenched solution was charged 15% w/v NaOH solution (12 ml) which resulted in the precipitation of a white solid. The slurry was stirred at room temperature for 30 minutes and the white solid was removed by filtration to give a clear organic filtrate which was dried with sodium sulfate (q.s.). The sodium sulfate was removed by filtration and the organic solution was concentrated under reduced pressure to afford intermediate B as a yellow liquid (20 g, 90%).

b. Synthesis of Intermediate C:

To a solution of formic acid (9.6 ml, 0.25 mol) and formaldehyde (7.1 ml, 0.25 mol) was charged intermediate B (10 g, 0.08 mol) and the reaction mixture was heated to reflux at 105 C for 16 hours. The reaction mixture was then cooled to room temperature and to the cooled solution was charged 10% sodium hydroxide solution followed by ethyl acetate (300 ml). The layers were separated and the organic layer was washed with brine (100 ml), then dried with sodium sulfate, filtered and the filtrate concentrated in vacuo to give intermediate C as a brown liquid (8.2 g, 66%).

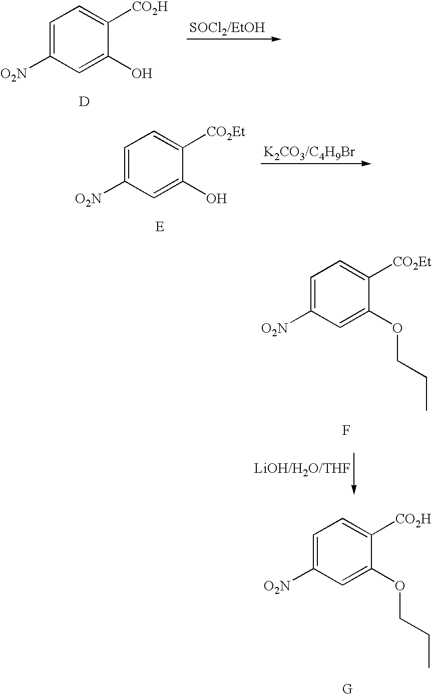

c. Synthesis of Intermediate E:

To a solution of anhydrous ethanol (500 ml) was charged intermediate D (50 g, 0.27 mol) and the reaction mixture was cooled to 0-5 C at which time thionyl chloride (59.6 ml, 0.81 mol) was slowly added to keep the reaction mixture temperature <20 C. After complete thionyl chloride addition the reaction mixture was heated to reflux and maintained at reflux overnight. The reaction mixture was cooled to 20 C and then concentrated to an oil by evaporation under reduced pressure. To the residue was charged a mixture of water (200 ml) and ethyl acetate (200 ml). The layers were separated and the aqueous was extracted with additional ethyl acetate (2×200 ml). The combined organic layers were washed with 10% sodium bicarbonate solution (200 ml), water (200 ml) and brine (200 ml) and then dried over sodium sulfate, filtered and concentrated under reduced pressure to give intermediate E as a yellow solid (51 g, 89%).

e. Synthesis of Intermediate F:

To a solution of DMF (250 ml) was charged intermediate E (25.5 g, 0.12 mol) followed by potassium carbonate (49.65 g, 0.36 mol) and the reaction mixture was cooled to 0 C at which time n-propylbromide (16.5 ml, 0.18 mol) was charged and the resulting slurry was stirred at room temperature for 24 hours. The reaction mixture was then diluted with ethyl acetate (250 ml), filtered through a bed of celite and the cake was washed with ethyl acetate (100 ml). The combined organic layers were washed with water (3×200 ml) then brine (200 ml) and the organic layer was dried with sodium sulfate and concentrated in vacuo to give intermediate F as a yellow solid (28 g, 92%).

f. Synthesis of Intermediate G:

To a solution of THF (150 ml) and water (150 ml) was charged intermediate F (30 g, 0.118 mol) and to this reaction mixture was charged a solution of lithium hydroxide (14.2 g, 0.59 mol) in 50 ml of water, and the resulting mixture was stirred at room temperature overnight. The reaction mixture was concentrated under reduced pressure to ⅓ its original volume and 1.5 N HCl solution was added until the pH of the solution was <6. The reaction mixture was extracted with ethyl acetate (2×200 ml) and the combined organic layer was washed with water (100 ml) and then brine (100 ml), then dried with sodium sulfate (q.s.), concentrated under reduced pressure to give intermediate G as a yellow solid (22 g, 82%).

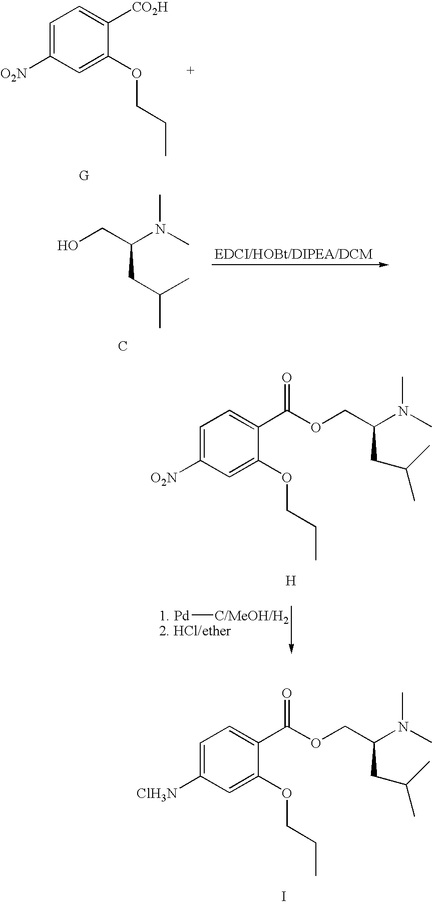

g. Synthesis of Intermediate H:

To a solution of dichloromethane (30 ml) was charged intermediate G (3.41 g, 0.12 mol), C (2 g, 0.013 mol), Hunig's base (7 ml, 0.041 mol), EDCI (3.97 g, 0.021 mol) and HOBt (0.38 g, 0.025 mol) and the reaction mixture was stirred at room temperature overnight. The reaction mixture was then diluted with water (50 ml) and extracted with dichloromethane (100 ml). The combined organic layers were washed with 10% sodium bicarbonate solution (50 ml), water (50 ml) and then brine (50 ml). The solution was then dried with sodium sulfate (q.s.), filtered and concentrated under reduced pressure to give a brown liquid which was purified by silica gel chromatography using 5% ethyl acetate/95% petroleum ether as eluent. The combined fractions were concentrated under reduced pressure to give intermediate H as a yellow liquid (1.2 g, 25%).

h. Synthesis of Compound 3 Hydrochloride Salt:

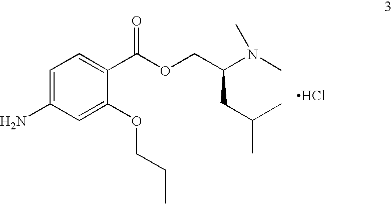

To a solution of methanol (50 ml) was charged intermediate H (1.2 g, 0.003 mol) followed by Pd—C (0.12 g, 10 mol %) and hydrogen gas was introduced and the reaction mixture was hydrogenated at 3.5 kg/cm2 for 2 hours. The reaction mixture was then filtered over celite and the filtrate was concentrated under reduced pressure and the residue was purified using silica gel chromatography eluting with 1.5% methanol in chloroform. The combined fractions were evaporated under reduced pressure to give Compound 3 free base as a brown liquid (0.37 g, 30%).

To a solution of chloroform (25 ml) was charged Compound 3 free base (0.44 g) and to this solution was charged HCl in diethylether (5 ml) and the reaction mixture was stirred at room temperature for 30 minutes. The solution was concentrated to dryness and diethyl ether was re-charged and the resulting solid was collected and washed with dichloromethane and ethyl acetate to yield the hydrochloride salt of Compound 3 as an off-white solid. HPLC purity (AUC): 91.4%; M+H+=323; 1H NMR (DMSO): δ (ppm)=0.9, m (6H); 1.0, t (3H); 1.8, m (5H); 2.7, s (3H); 2.8, s (3H); 3.7, bs (1H); 4.0, t (2H); 4.5, m (2H); 6.4, d (1H); 6.5, s (1H); 7.8, d (1H).

### Example 4

### General Synthesis of Representative Compounds

Other representative compounds of formula I can be prepared using the following general synthetic scheme.

a. Synthesis of Substituted Amino Alcohol Coupling Pieces:

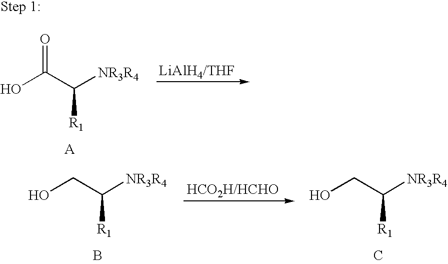

To a solution of a polar non protic solvent such as THF is charged a hydride source, such as lithium aluminum hydride (0.5 to 2.0 equivalents) followed by an amino acid, such as L-Leucine, L-isoleucine, L-phenyalanine (A) and the mixture is refluxed for 12-24 hours until reduction is complete. This mixture is cooled to 0-10 C, diluted with a polar non protic solvent such as diethyl ether and is quenched with water and basified with a sodium hydroxide solution, such as 15% sodium hydroxide solution. The resulting solid is washed with this polar non protic solvent, dried with magnesium or sodium sulfate and concentrated in vacuo to afford the product (B) as a liquid.

b. Synthesis of Piperidinyl Containing Amino Alcohol Intermediates

To a solution of compound B is charged an alcoholic solvent such as ethanol, followed by potassium or sodium carbonate followed by addition of 1,5-dibromopentane and the reaction mixture is heated to reflux for 48-72 hours, then cooled to room temperature and filtered through a bed of celite. Concentration of the organic layer in vacuo gives an oil which is purified by silica gel chromatography. The fractions containing pure product are combined and the solvent removed in vacuo to provide the piperidinyl compounds.

c. Synthesis of Dimethyl or Dimethyl Containing Amino Alcohol Intermediates—Dimethyl Compound Procedure Provided Below.

To a solution of formic acid and formaldehyde is charged compound B and the reaction mixture is heated to reflux for 16-24 hours and then is cooled to room temperature at which time a sodium hydroxide solution is charged followed by an organic solvent such as ethyl acetate. After washing of the organic layer with brine and drying with sodium or magnesium sulfate and filtration, the filtrate is concentrated in vacuo to give C.

d. Synthesis of Common Intermediate G:

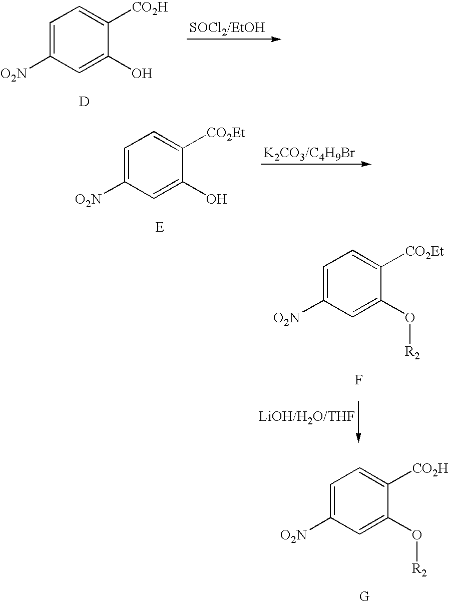

To a solution of a protic polar solvent, such as ethanol, is charged 2-hydroxy-4-nitrobenzoic acid followed by a chlorinating agent such as thionyl chloride. After reflux for 8-16 hours the reaction mixture is concentrated and extracted with an organic solvent such as ethyl acetate, washed with sodium carbonate solution, water and brine and the organic evaporated in vacuo to afford the product as a yellow solid.

e. To a solution of a polar non-protic solvent such as DMF is charged Compound E and potassium carbonate followed by an alkyl bromide (such as n-ethyl, n-propyl or n-butylbromide) and the reaction mixture is stirred at room temperature for 16-30 hours. The reaction mixture is diluted with an organic solvent, such as ethyl acetate, and is washed with water and brine and concentrated in vacuo to afford the product as a yellow solid.
f. To a solution of a polar non-protic organic solvent such as THF and water is charged Compound F and an alkyl hydroxide, such as lithium hydroxide, and the solution is stirred at room temperature for 8-16 hours. The reaction mixture is concentrated, acidified with an HCl solution, such as 1.5 M HCl solution and is extracted with an organic solvent such as ethyl acetate. The organic solution is washed with water and brine and dried over magnesium or sodium sulfate and concentrated in vacuo to afford the product as an off-white solid.
g.

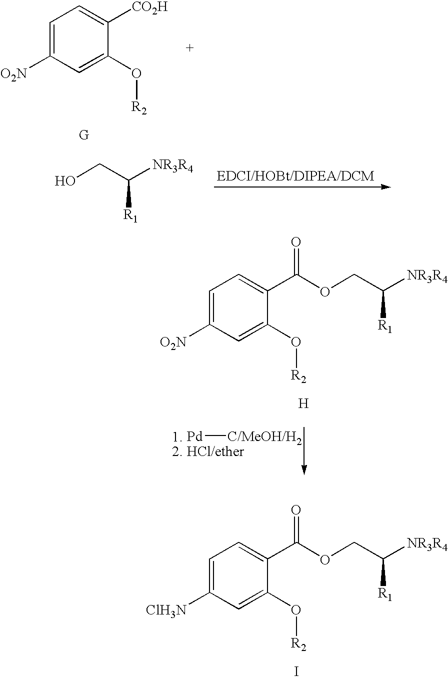

To a solution of a non-polar organic solvent is charged G, an amino alcohol, EDCI, HOBt and an amine base, (e.g. Hunig's base), and the reaction mixture is allowed to stir overnight at room temperature. The reaction mixture is then diluted with water, extracted with a non-polar organic solvent and washed with bicarbonate solution, brine and dried with magnesium or sodium sulfate. Purification by silica gel chromatography gives the product H as an oil.

h. To a solution of an alcoholic solvent, preferably methanol, is charged H, a Pd—C catalyst and hydrogen gas is introduced and the reaction mixture stirred for several hours. The reaction mixture is filtered over celite, the filtrate is evaporated under reduced pressure and the residue is purified by silica gel chromatography to give the free base of compound I. The free base is dissolved in a non-polar organic solvent and HCl in a non-polar organic solvent is introduced. The solution is concentrated to dryness and an organic solvent, such as diethyl ether, is charged resulting in precipitation of a solid which is collected to afford compound I as its hydrochloride salt.

### Example 5

### Synthesis of Representative Compounds 4, 5, and 6

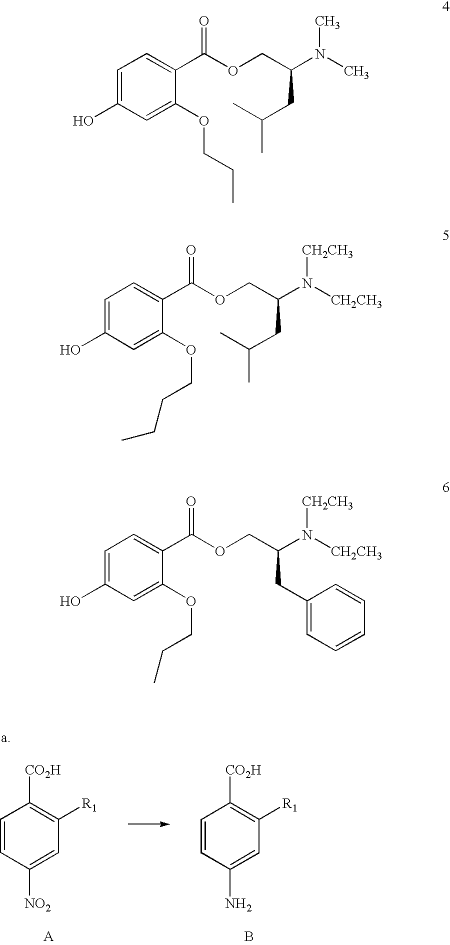

To a 500 ml flask was charged A, 4-nitro-2-R₁-benzoic acid [where R₁ is propoxy or butoxy] (10 g, 0.044 mol), followed by methanol (100 ml) and to the resulting solution was charged 10% Pd—C (1 g) and the flask was pressurized with 4 kg/cm² of hydrogen and the reaction mixture was allowed to stir at room temperature overnight. After reaction completion was confirmed by TLC analysis the reaction mixture was filtered over celite and the filtrate was concentrated in vacuo to give compound B as a brown solid (7.99 grams, 91% yield).

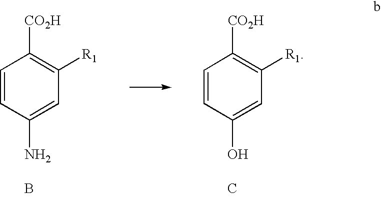

To a 500 ml round bottom flask was charged B (8.1 g, 0.042 mol) followed by concentrated sulfuric acid (162 ml) and water (162 ml) and the reaction mixture was heated to 80° C. at which time a pre-made solution of sodium nitrite (3.9 g, 0.062 mol) was charged in portions. After stirring at this temperature for 3-4 hours, reaction completion was confirmed by TLC analysis, the mixture was cooled to 20° C. and then poured over ice (q.s.) followed by extraction with ethyl acetate. The organic was washed with water and then brine and dried with sodium sulfate (q.s.) and then concentrated in vacuo to give a solid which was purified by silica gel chromatography (9:1 chloroform:methanol) to give compound C as a red solid (7.5 g, 92%).

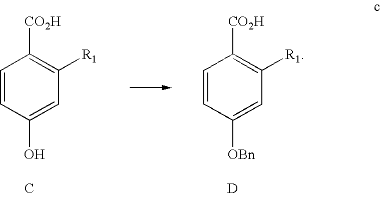

Into a 250 ml round bottom flask was charged compound C (7.4 g, 0.038 mol) followed by ethanol (70 ml) and water (7 ml), benzyl bromide (7.05 g, 0.042 mol) and potassium hydroxide (4.23 g, 0.076 mol) and the mixture was heated to reflux for 18 hours until reaction completion was confirmed by TLC analysis. The reaction mixture was cooled to room temperature and the ethanol was removed in vacuo to ⅕ the original volume. The solution was neutralized with 1.5 N HCl and then extracted with ethyl acetate. The organic was washed with water and then brine and dried with sodium sulfate then concentrated in vacuo to give crude compound D. Compound D was purified by silica gel chromatography using 5:1 petroleum ethyl/ethyl acetate to give compound D as a pale yellow solid (3.0 g, 27% yield).

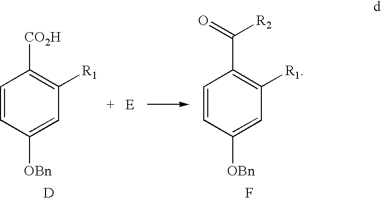

Into a 100 ml round bottom flask was charged dichloromethane (25 ml) followed by compound D (1.1 g, 0.0038 mol), compound E [N,N-dimethylleucinol, N,N diethylleucinol or N,N diethylphenylalaminol] (0.95 eq), Hunig's base (1.36 g, 0.0105 mol), EDC (1.0 g, 0.0053 mol), and HOBt (0.1 g, 0.0009 mol) and the reaction mixture was stirred overnight at room temperature until TLC analysis confirmed reaction completion. The reaction mixture was concentrated in vacuo, ethyl acetate was charged to the residue and the resulting solution was washed with water and brine, dried with sodium sulfate and concentrated in vacuo to give crude compound F. The crude material was purified by silica gel chromatography (7:3 petroleum ether/ethyl acetate) to give compound F as a yellow liquid (0.15 g, 10%).

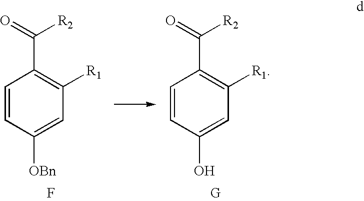

Into a 50 ml round bottom flask was charged methanol (10 ml) followed by Pd—C (25 mg) and the mixture was hydrogenated overnight at room temperature under 4 kg/cm² of hydrogen pressure until TLC analysis showed reaction completion. The reaction mixture was filtered over celite and the filtrated was concentrated under reduced pressure to give crude compound 4, which was purified by silica gel:chromatography (9:1 chloroform/methanol) to provide a yellow liquid. Compounds 5 and 6 were prepared using a similar procedure.

### Example 6

### Synthesis of Representative Compounds 7 and 8

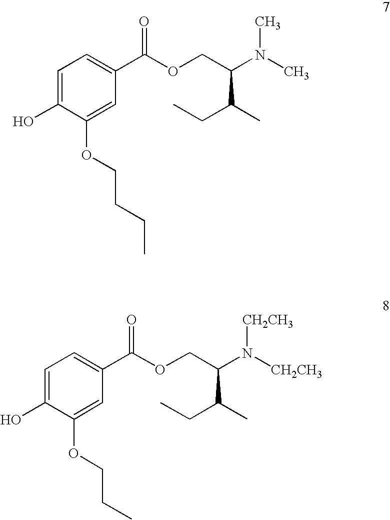

Compounds 7 and 8 can also be prepared using the following synthetic scheme wherein R₁ is propyl or butyl and R₂ is N,N-dimethylisoleucinol or N,N-diethylisoleucinol.

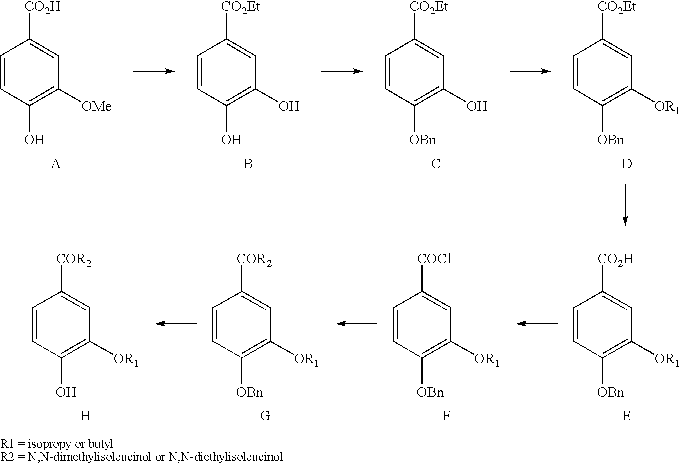

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the allowed claims.

All publications and patents cited herein are incorporated by reference in their entirety.

1-2. (canceled)

3. A compound of formula IIa or IIb: 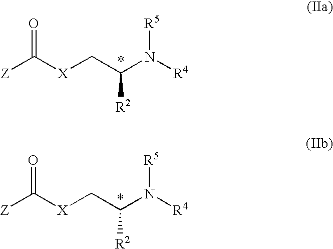
or a prodrug, salt, hydrate, solvent or N-oxide thereof,
wherein:
Z is a group of the following formula: 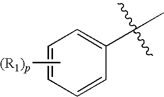
each R¹ is independently hydrogen, —F, —Cl, —Br, —I, —R⁶, —OR⁶, —SR⁶, —NR⁶R⁷ or —C(O)NR⁶R⁷;
p is an integer from 1 to 3;
X is —O— or —S—;
R² is the sidechain of an amino acid; or is selected from methyl, 3-guanidinopropyl, aminocarbonylmethyl, carboxymethyl, mercaptomethyl, 2-carboxy-2-aminoethyldithiomethyl, 2-carboxyethyl, 2-(aminocarbonyl)ethyl, imidazolylmethyl, 4-amino-3-hydroxybutyl, 4-aminobutyl, 2-(methylthio)ethyl, hydroxymethyl, 1-hydroxyethyl, indolylmethyl, 4-hydroxybenzyl, isopropyl, 2-methylpropyl, 1-methylpropyl, and benzyl; or is (C3-C6) alkyl;
R⁴ and R⁵ are independently H, (C1-C6) alkyl optionally substituted with one or more of the same or different R⁸ groups or optionally R⁴ and R⁵ together with the nitrogen atom to which they are bonded form a cycloheteroalkyl ring;
R⁶ and R⁷ are independently hydrogen, alkyl, cycloalkyl, cycloheteroalkyl, aryl or heteroaryl; and
R⁸ is —F, —Cl, —Br, —I, —R⁶, —OR⁶, —SR⁶, —NR⁶R⁷, —CF₃, —CN, —C(O)R⁶, —C(O)OR⁶, —C(O)SR⁶, —C(O)NR⁶R⁷, or aryl which is optionally substituted with one or more —F, —Cl, —Br, —I, —R⁶, —OR⁶, —SR⁶, —NR⁶R⁷, —CF₃, —CN, —C(O)R⁶, —C(O)OR⁶, —C(O)SR⁶, or —C(O)NR⁶R⁷;
wherein the center marked by * in the formula has the absolute configuration shown provided that the compound is not:

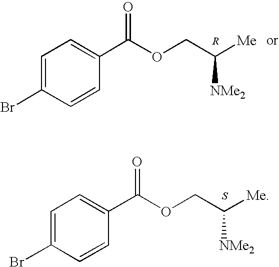

4. (canceled)

5. The compound of claim 3 wherein Z is:

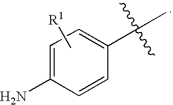

6-7. (canceled)

8. The compound of claim 3 wherein Z is:

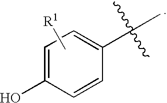

9-13. (canceled)

14. The compound of claim 3 wherein R² is the sidechain of a natural amino acid.

15. The compound of claim 3 wherein R² is methyl, isopropyl, 2-methylpropyl, 1-methylpropyl, or benzyl.

16-17. (canceled)

18. The compound of claim 3 wherein R⁴ and R⁵ are each independently methyl or ethyl.

19. The compound of claim 3 wherein R⁴ and R⁵ together with the nitrogen atom to which they are bonded form a piperadino ring.

20. The compound of claim 3 wherein X is O.

21. A compound having the structure:

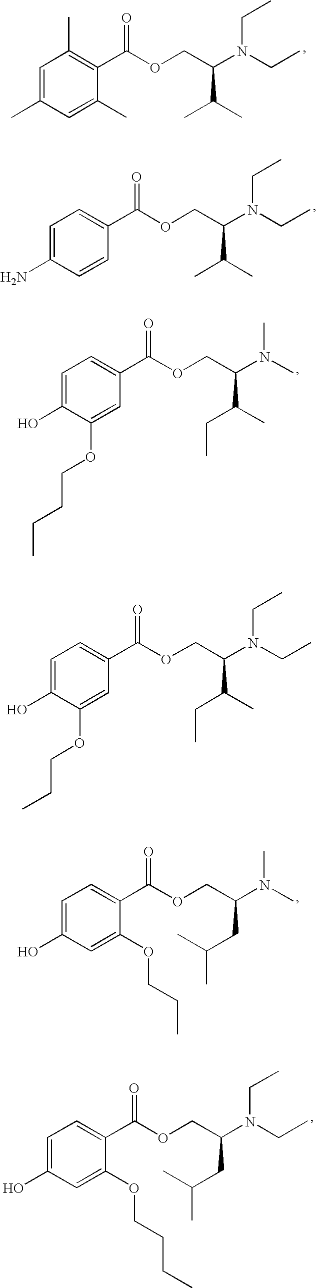

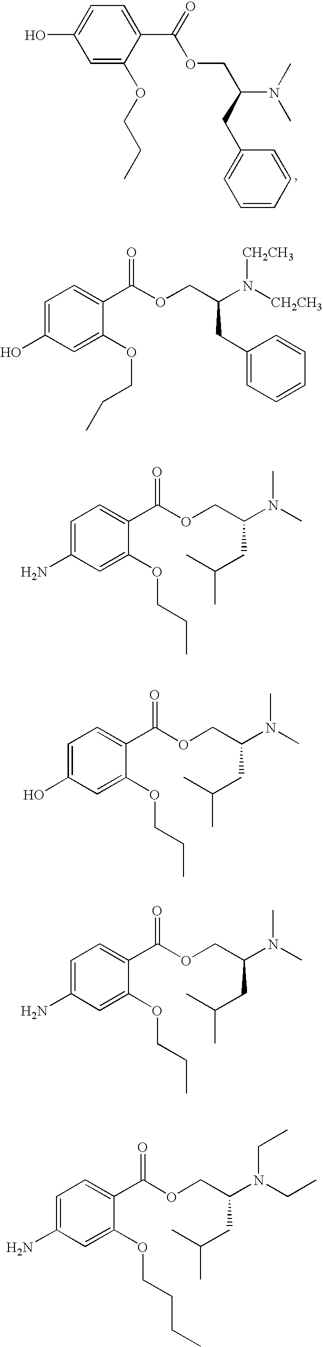

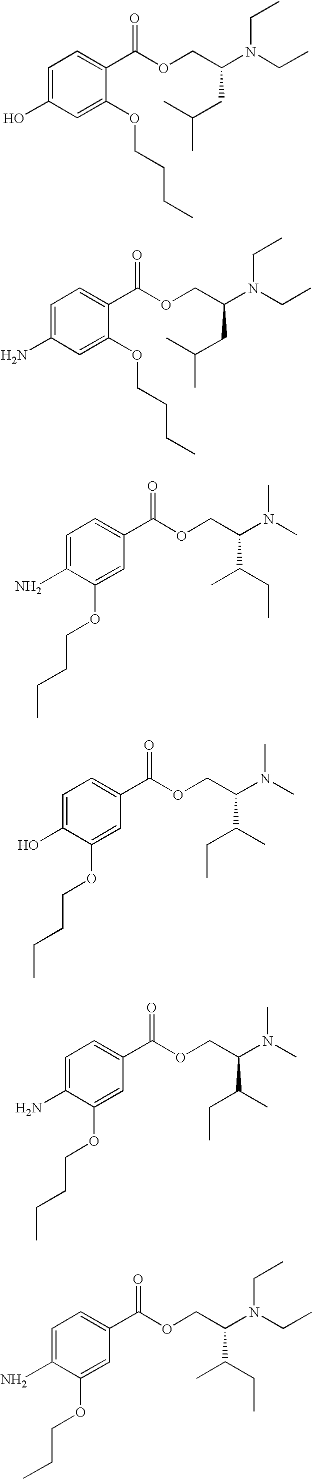

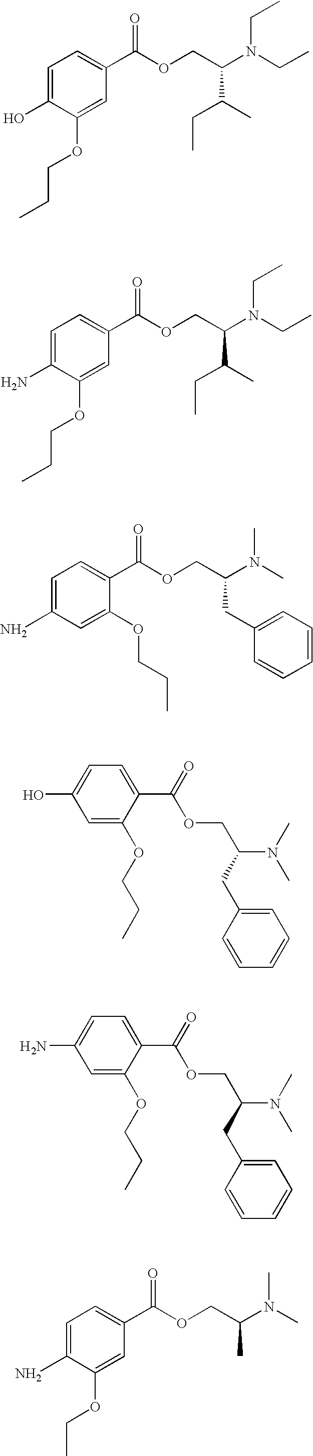

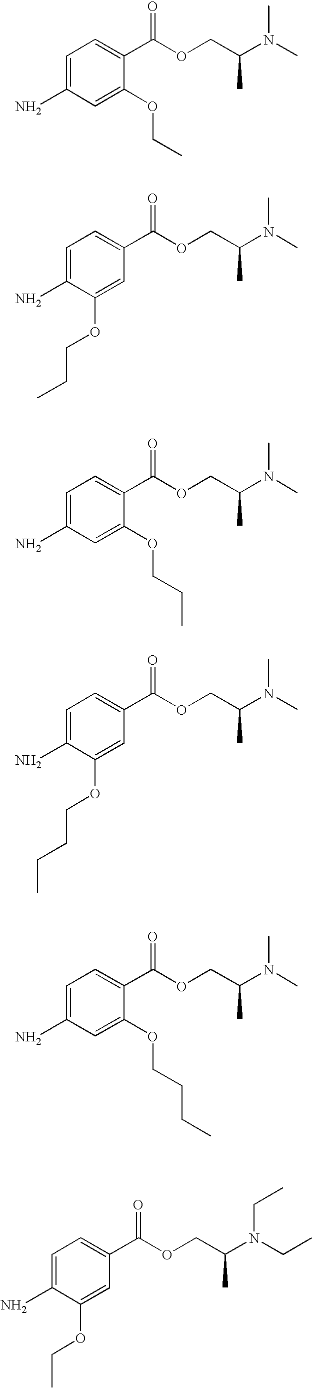

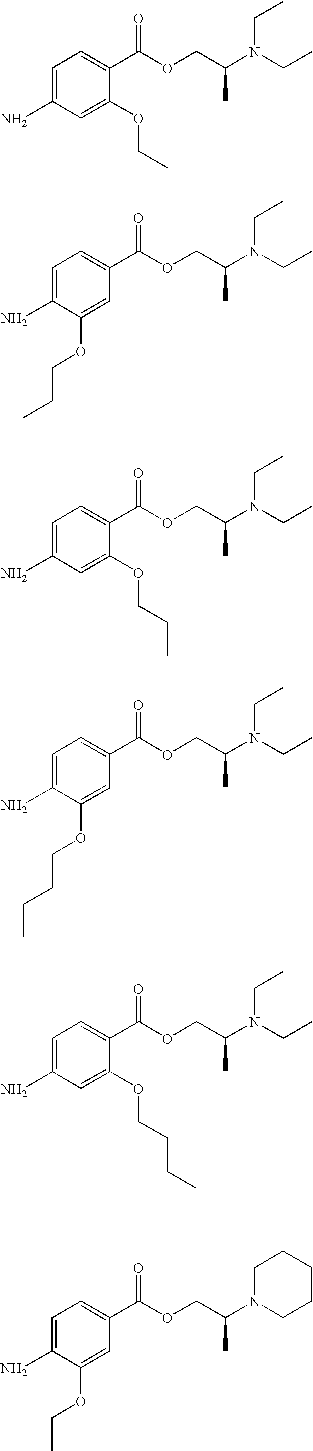

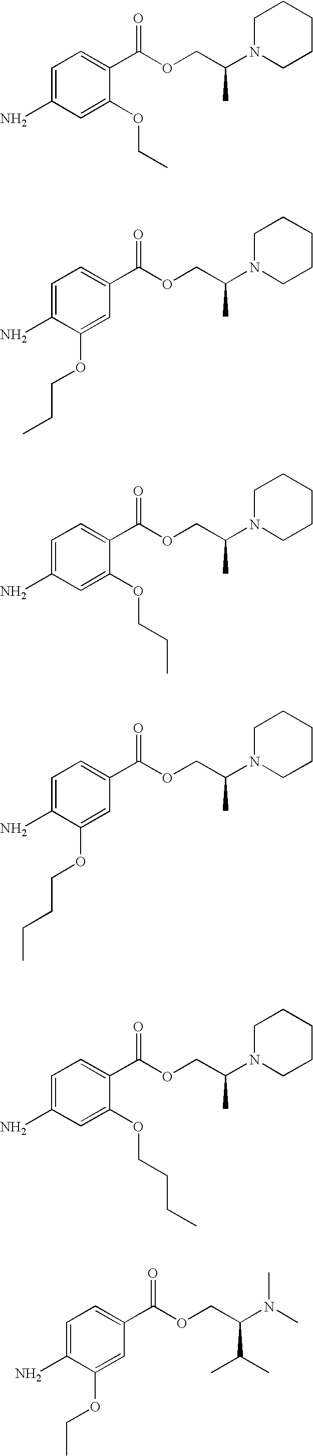

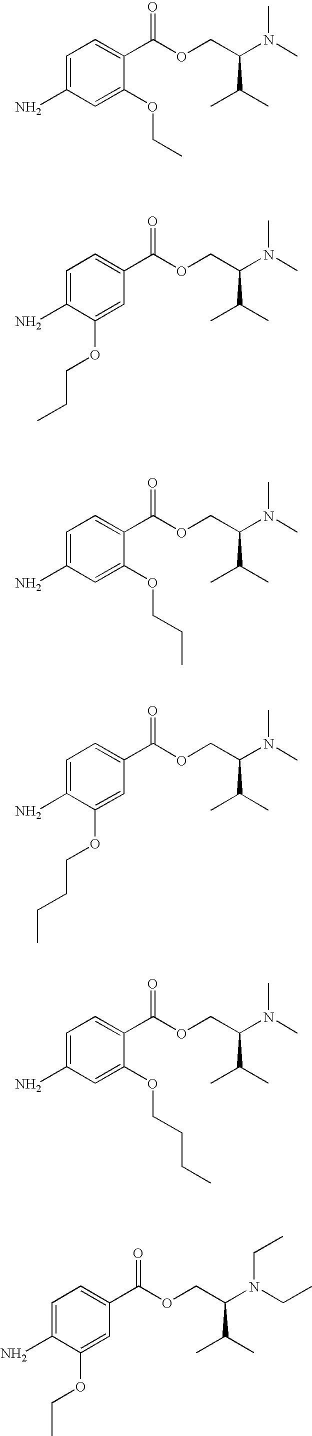

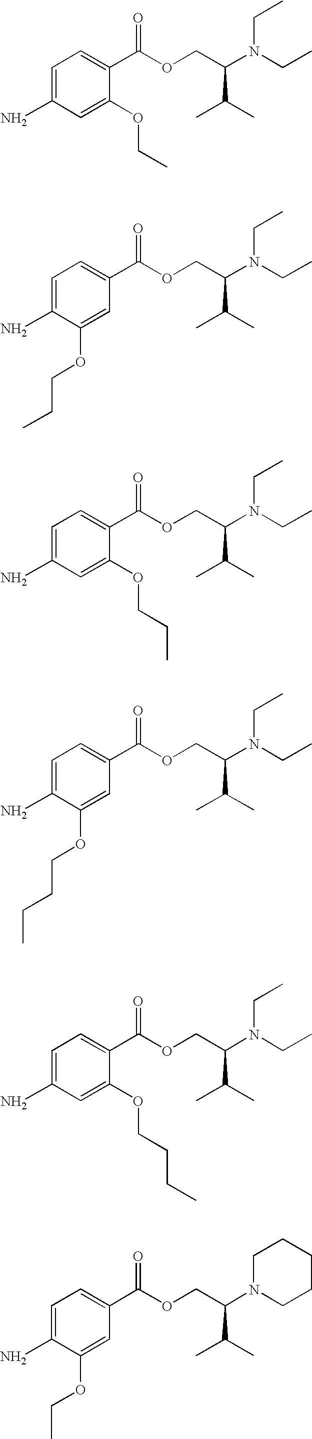

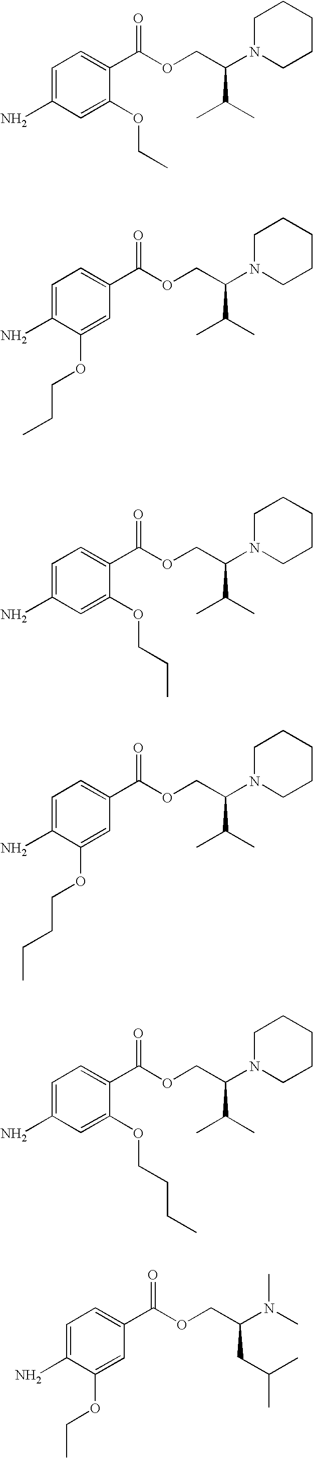

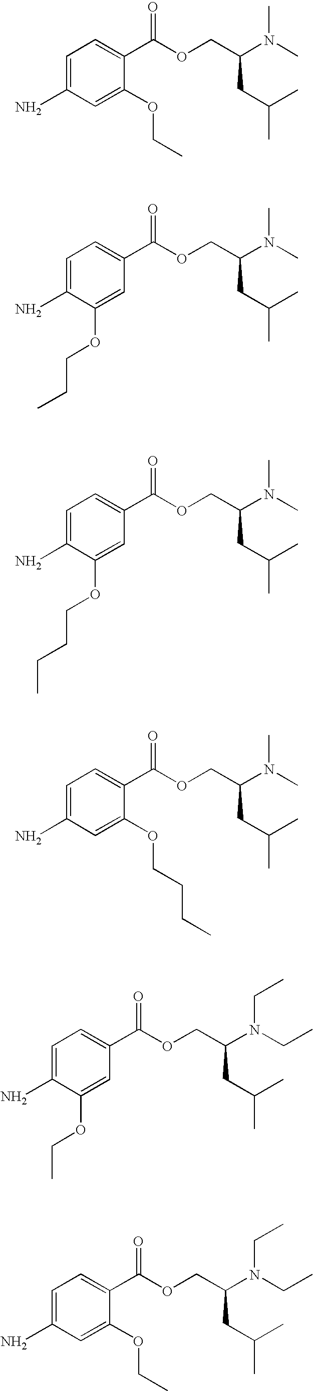

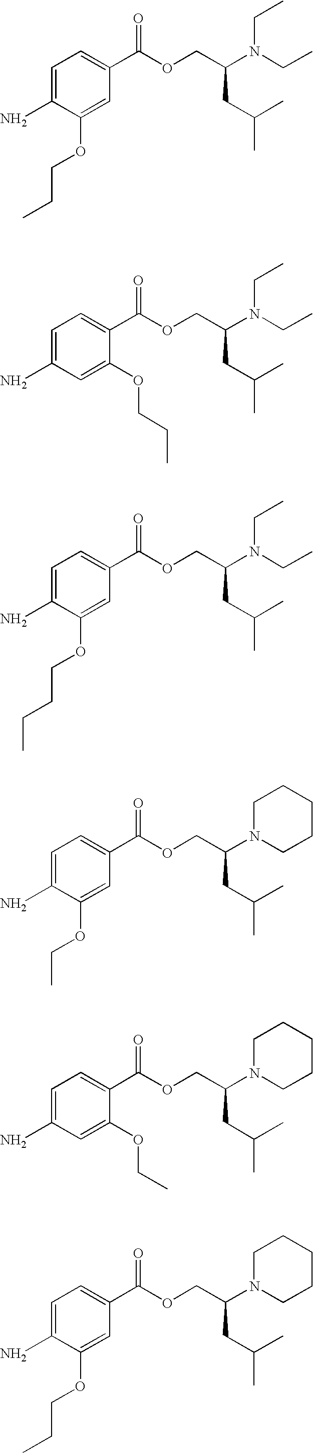

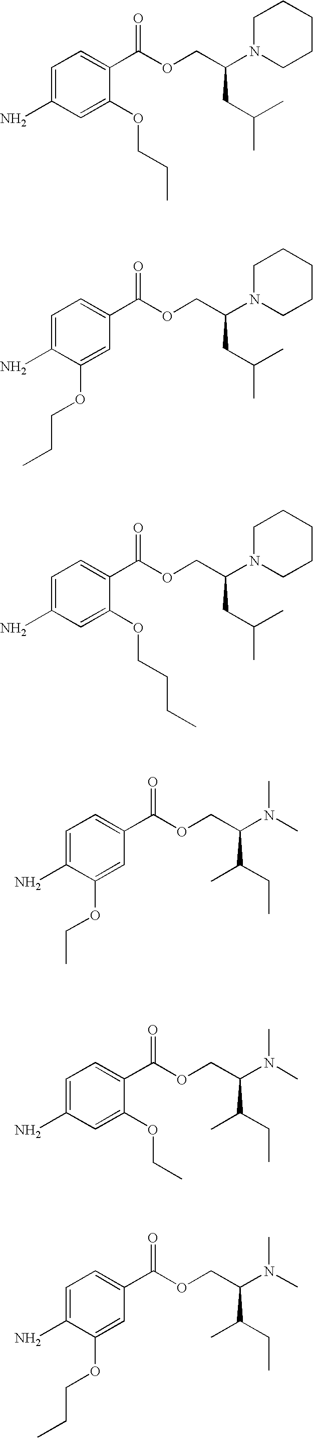

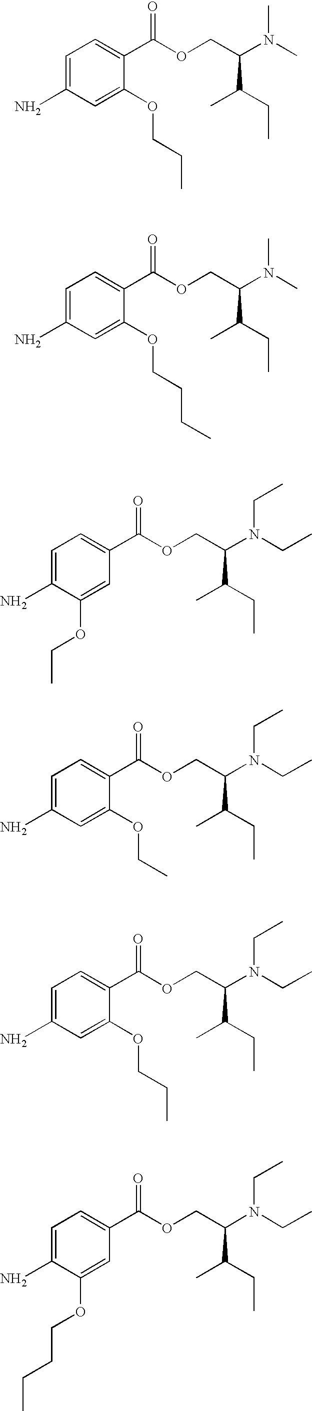

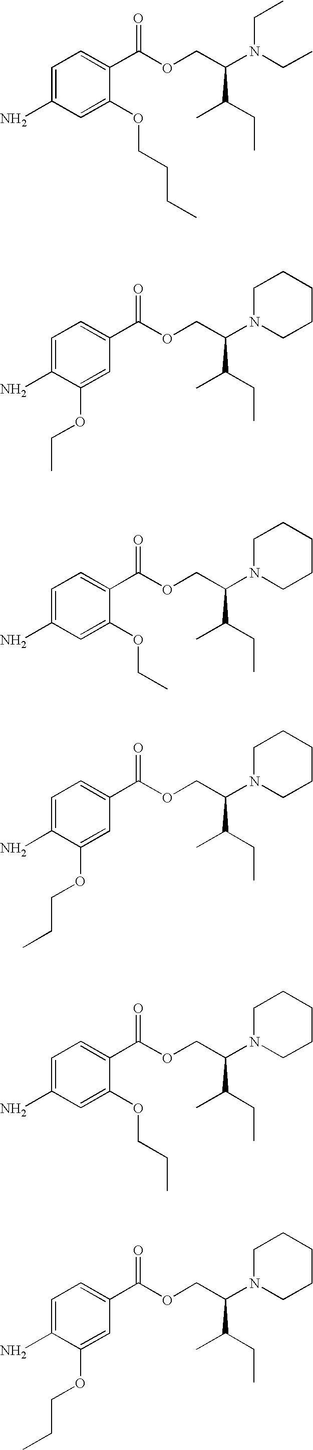

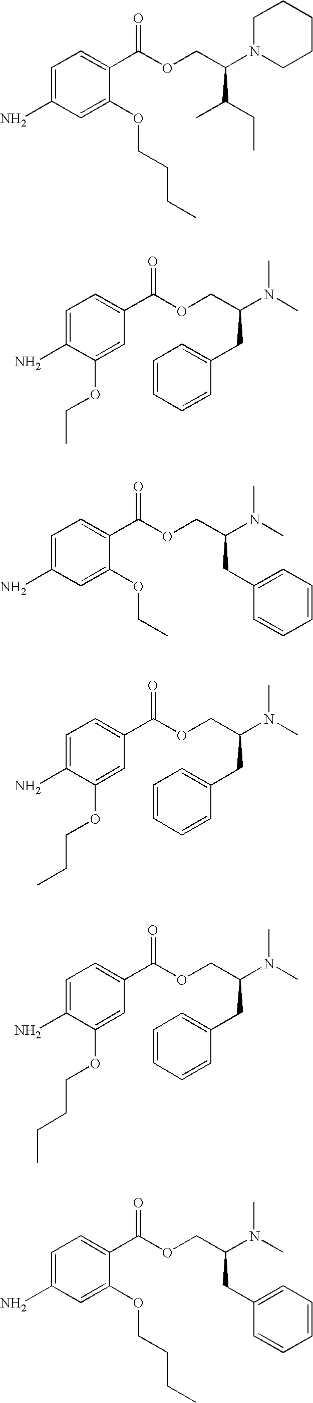

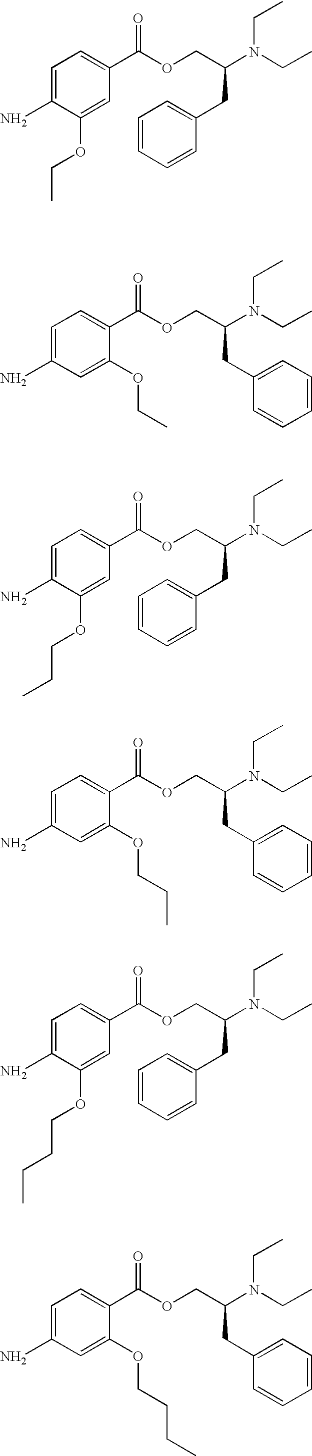

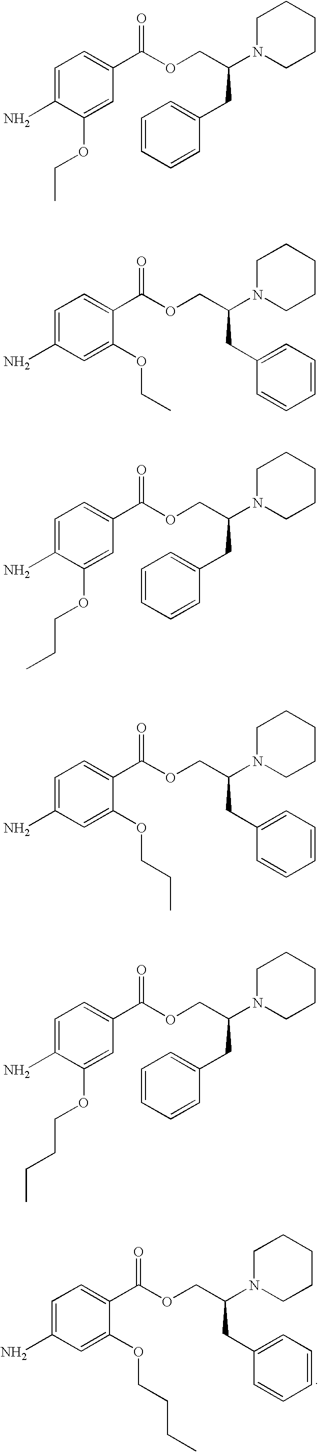

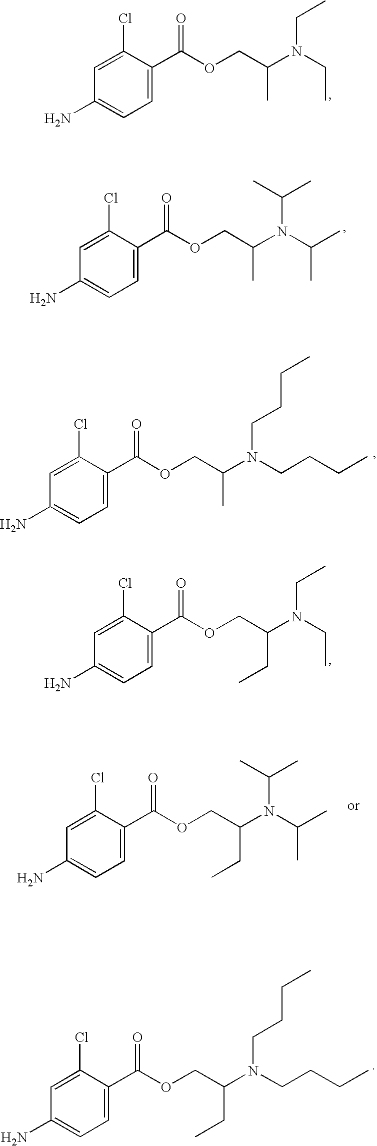

22. A compound having the structure:

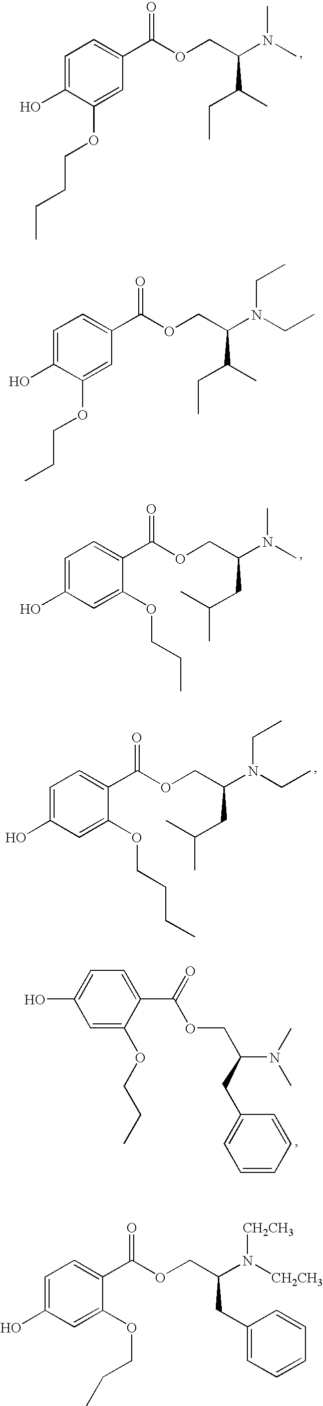

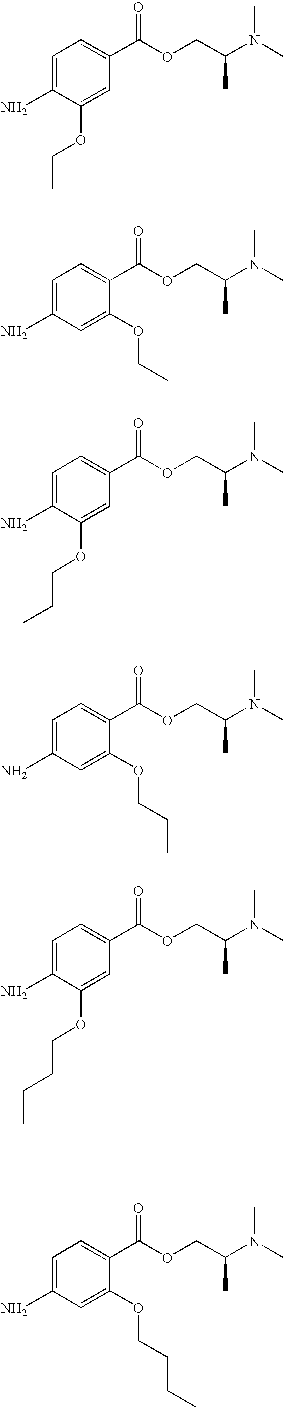

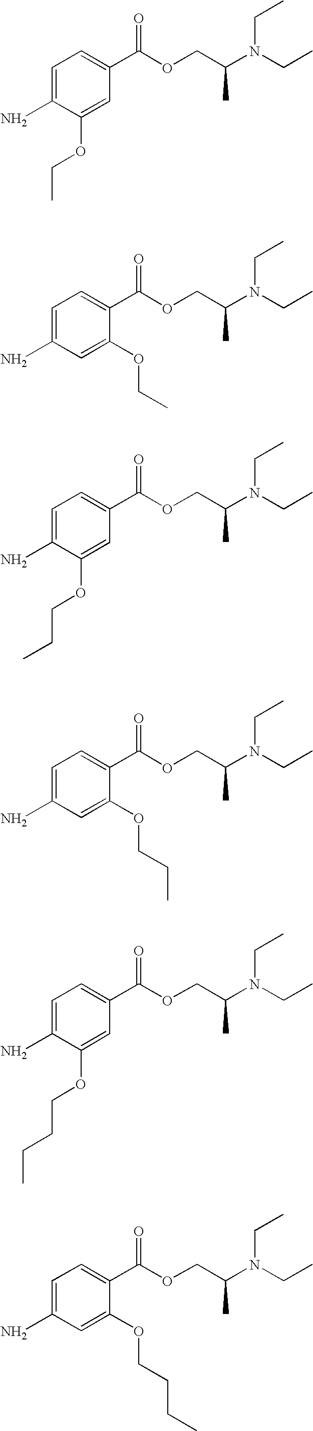

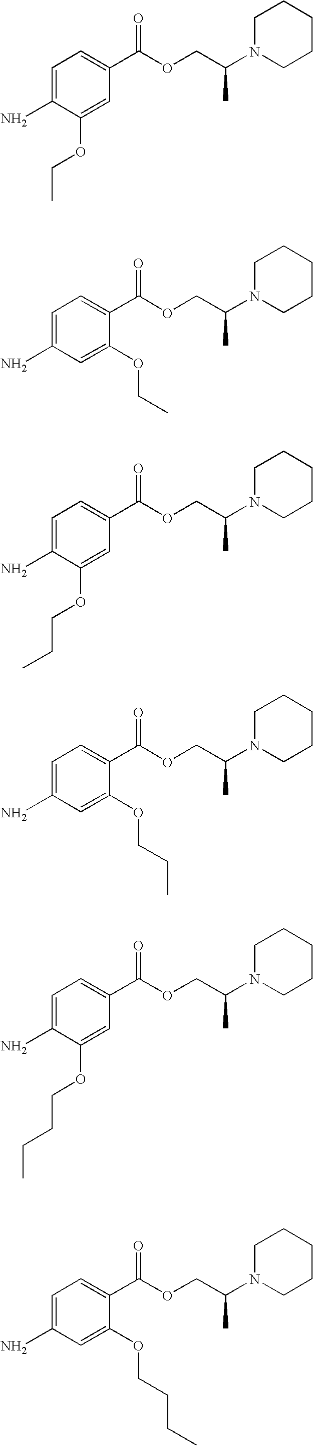

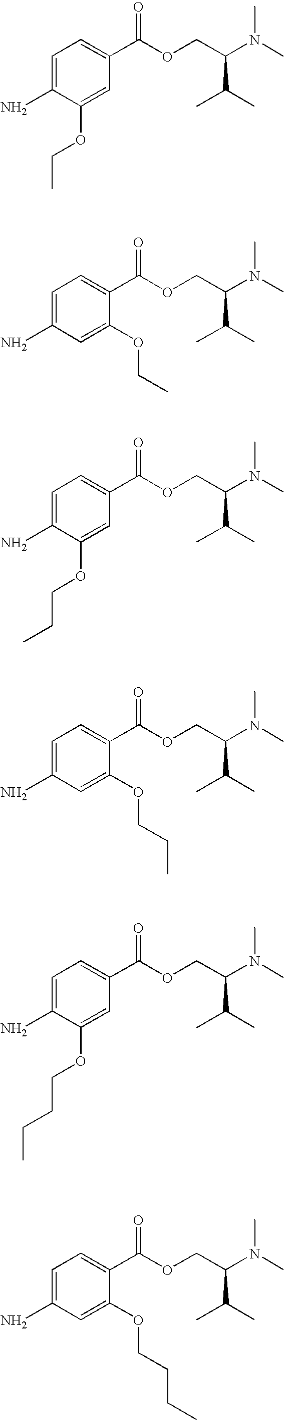

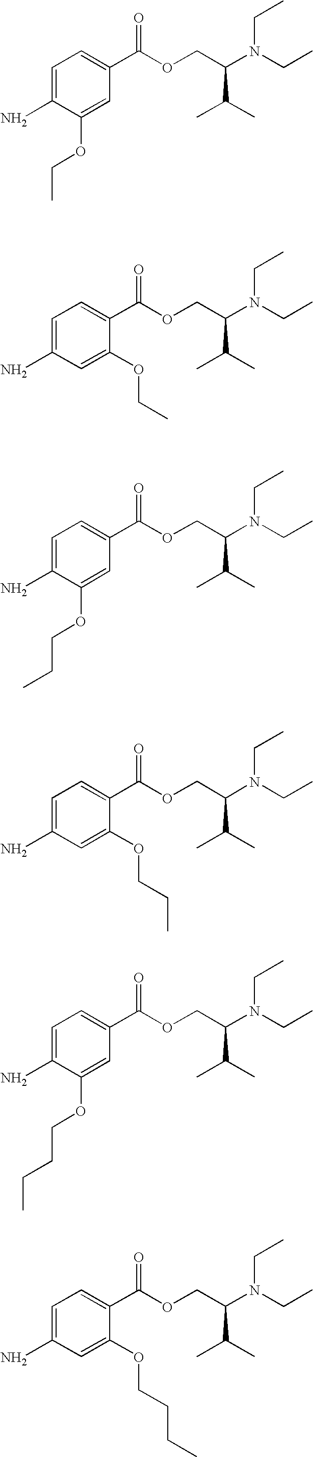

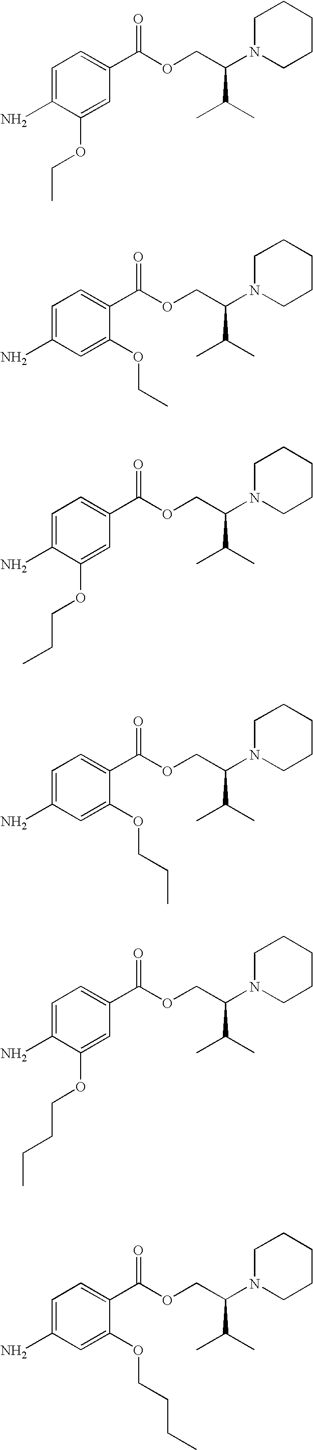

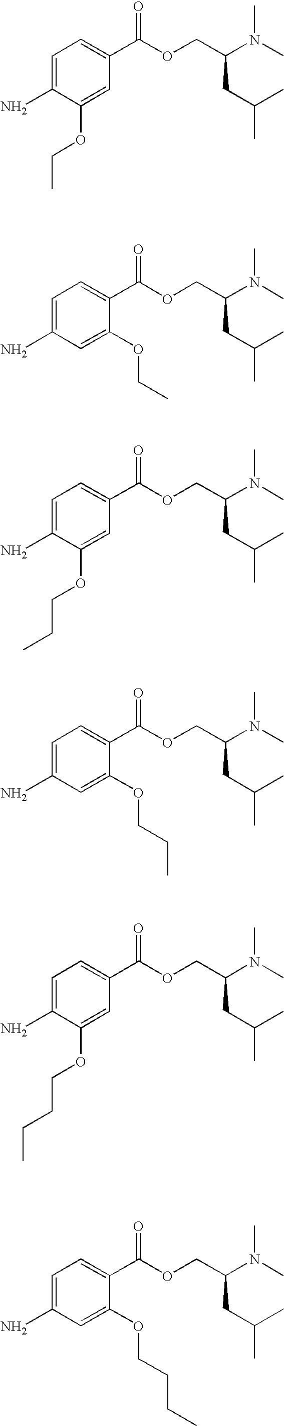

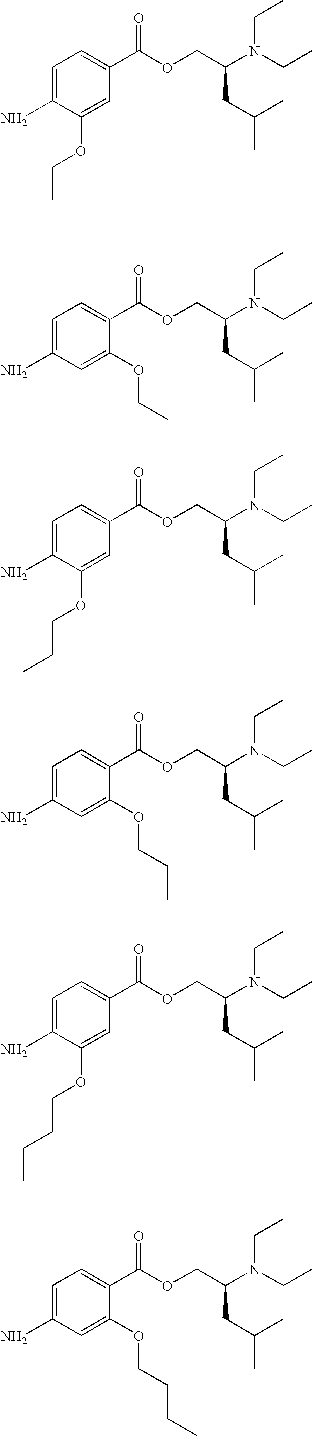

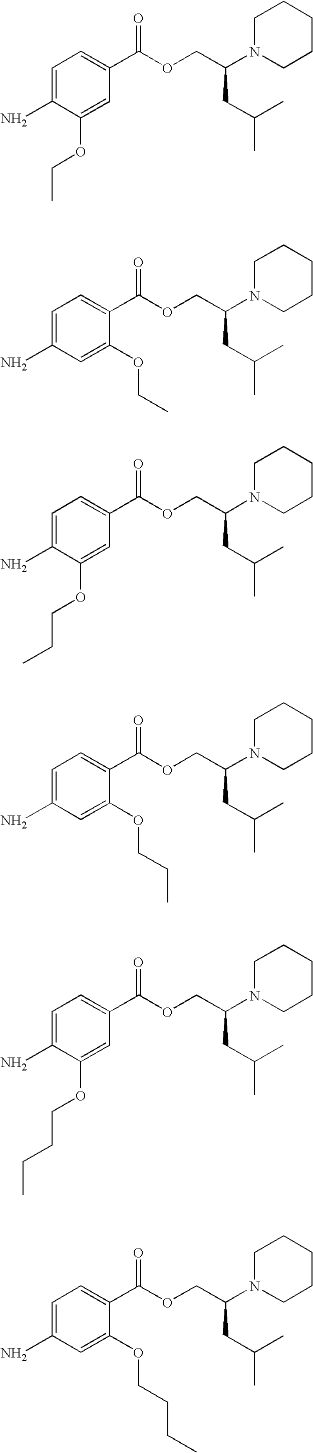

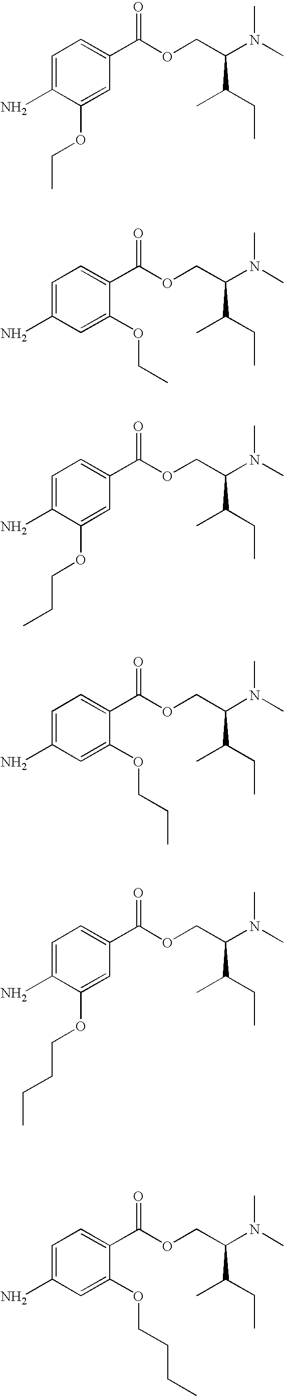

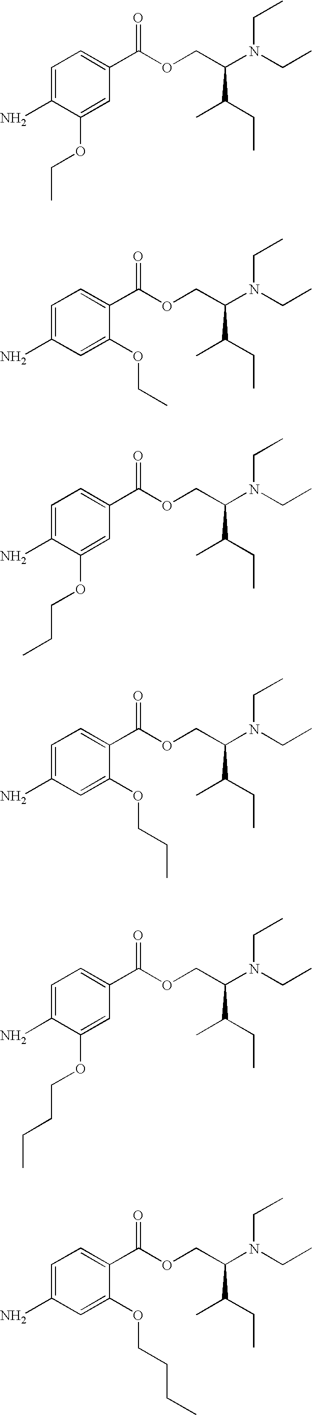

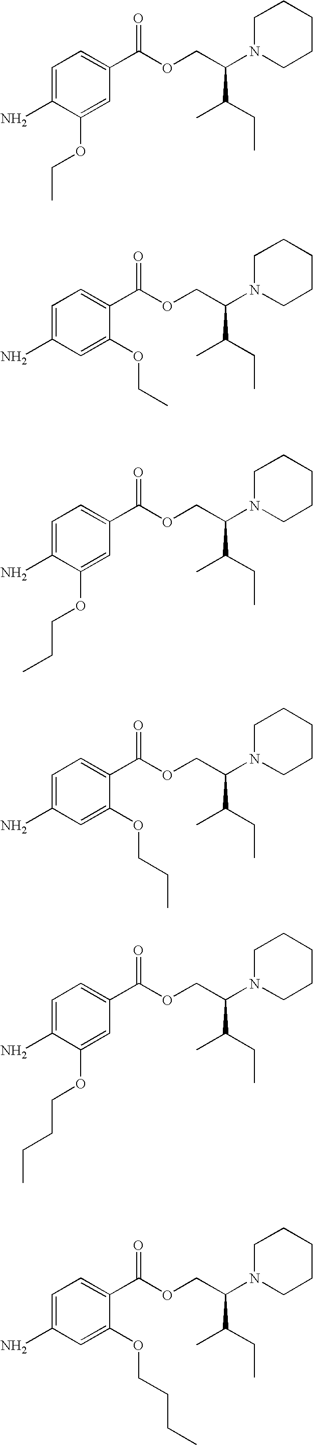

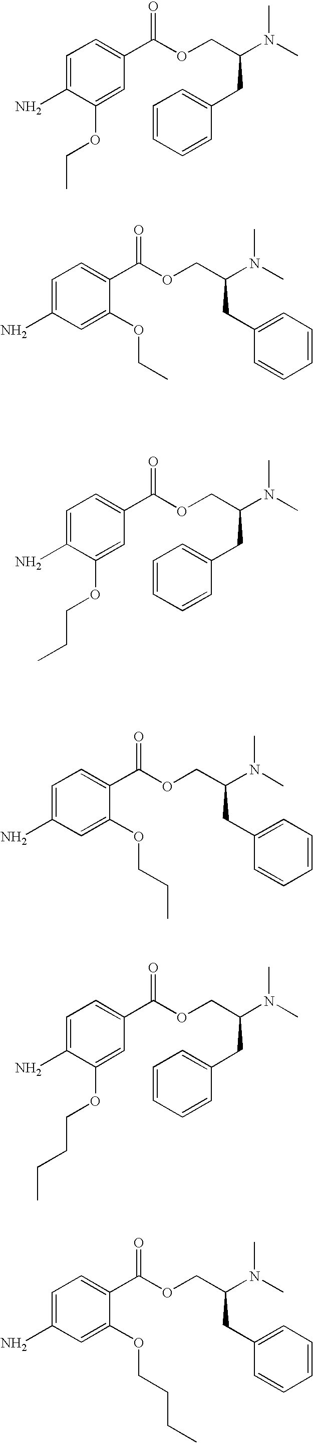

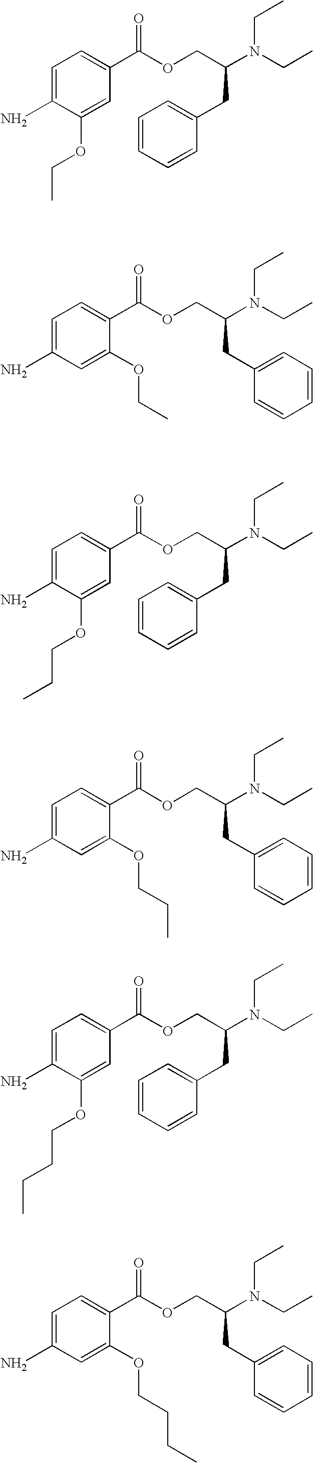

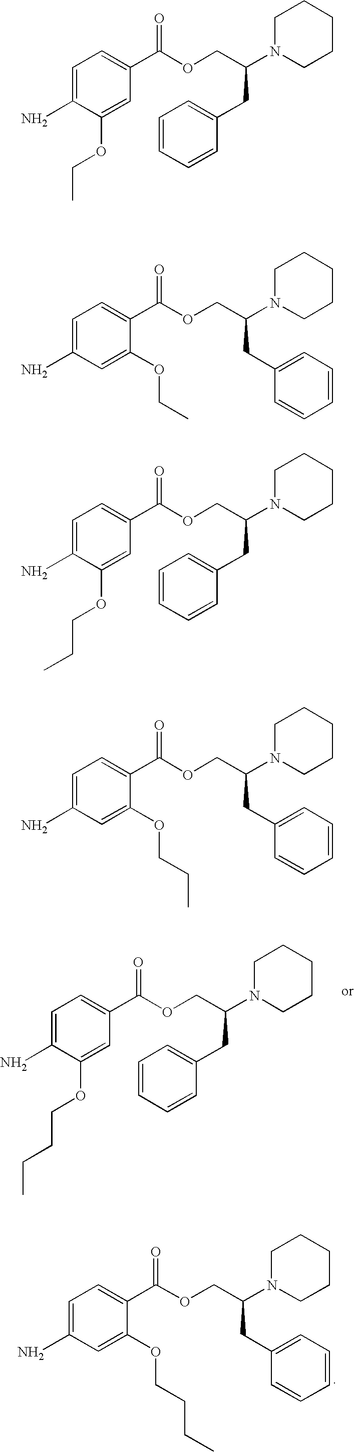

23-27. (canceled)

28. The compound of claim 3 wherein R² is (C3-C6) alkyl.

29. The compound of claim 3 wherein R² is propyl, isopropyl, butyl, isobutyl, secbutyl, pentyl, isopentyl, secpentyl, or hexyl.

30. A pharmaceutical composition comprising a compound of claim 3 and a pharmaceutically acceptable vehicle.

31. (canceled)

32. A method of inducing or maintaining local anesthesia in a patient comprising administering to the patient a compound of claim 3.

33. A method of inducing or maintaining local anesthesia in a patient comprising administering to the patient in need thereof the pharmaceutical composition of claim 30.

34. A method of treating or preventing pain in a patient comprising administering to the patient in need thereof a compound of claim 3.

35-37. (canceled)

38. The compound of claim 3, wherein Z is: 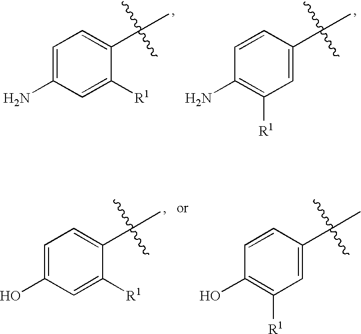
wherein R¹ is ethoxy, propoxy or butoxy.

39. The compound of claim 3, wherein R² is selected from methyl, 3-guanidinopropyl, aminocarbonylmethyl, carboxymethyl, mercaptomethyl, 2-carboxy-2-aminoethyldithiomethyl, 2-carboxyethyl, 2-(aminocarbonyl)ethyl, imidazolylmethyl, 4-amino-3-hydroxybutyl, 4-aminobutyl, 2-(methylthio)ethyl, hydroxymethyl, 1-hydroxyethyl, indolylmethyl, 4-hydroxybenzyl, isopropyl, 2-methylpropyl, 1-methylpropyl, and benzyl.

40. A compound of formula IIa: 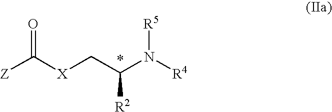
or a prodrug, salt, hydrate, solvent or N-oxide thereof,
wherein:
Z is a group of the following formula: 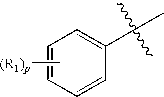
each R¹ is independently hydrogen, —F, —Cl, —I, —R⁶, —OR⁶, —SR⁶, —NR⁶R⁷ or —C(O)NR⁶R⁷;
p is an integer from 1 to 3;
X is —O— or —S—;
R² is the sidechain of an amino acid; or is selected from methyl, 3-guanidinopropyl, aminocarbonylmethyl, carboxymethyl, mercaptomethyl, 2-carboxy-2-aminoethyldithiomethyl, 2-carboxyethyl, 2-(aminocarbonyl)ethyl, imidazolylmethyl, 4-amino-3-hydroxybutyl, 4-aminobutyl, 2-(methylthio)ethyl, hydroxymethyl, 1-hydroxyethyl, indolylmethyl, 4-hydroxybenzyl, isopropyl, 2-methylpropyl, 1-methylpropyl, and benzyl; or is (C3-C6) alkyl;
R⁴ and R⁵ are independently H, (C1-C6) alkyl optionally substituted with one or more of the same or different R⁸ groups or optionally R⁴ and R⁵ together with the nitrogen atom to which they are bonded form a cycloheteroalkyl ring;
R⁶ and R⁷ are independently hydrogen, alkyl, cycloalkyl, cycloheteroalkyl, aryl or heteroaryl; and
R⁸ is —F, —Cl, —Br, —I, —R⁶, —OR⁶, —SR⁶, —NR⁶R⁷, —CF₃, —CN, —C(O)R⁶, —C(O)OR⁶, —C(O)SR⁶, —C(O)NR⁶R⁷, or aryl which is optionally substituted with one or more —F, —Cl, —Br, —I, —R⁶, —OR⁶, —SR⁶, —NR⁶R⁷, —CF₃, —CN, —C(O)R⁶, —C(O)OR⁶, —C(O)SR⁶, or —C(O)NR⁶R⁷;
wherein the center marked by * in the formula has the absolute configuration shown.

41. A compound of formula IIb: 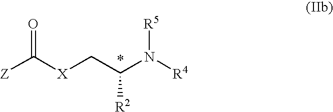
or a prodrug, salt, hydrate, solvent or N-oxide thereof,
wherein:
Z is a group of the following formula: 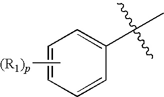
each R¹ is independently hydrogen, —F, —Cl, —I, —R⁶, —OR⁶, —SR⁶, —NR⁶R⁷ or —C(O)NR⁶R⁷;
p is an integer from 1 to 3;
X is —O— or —S—;
R² is the sidechain of an amino acid; or is selected from methyl, 3-guanidinopropyl, aminocarbonylmethyl, carboxymethyl, mercaptomethyl, 2-carboxy-2-aminoethyldithiomethyl, 2-carboxyethyl, 2-(aminocarbonyl)ethyl, imidazolylmethyl, 4-amino-3-hydroxybutyl, 4-aminobutyl, 2-(methylthio)ethyl, hydroxymethyl, 1-hydroxyethyl, indolylmethyl, 4-hydroxybenzyl, isopropyl, 2-methylpropyl, 1-methylpropyl, and benzyl; or is (C3-C6) alkyl;
R⁴ and R⁵ are independently H, (C1-C6) alkyl optionally substituted with one or more of the same or different R⁸ groups or optionally R⁴ and R⁵ together with the nitrogen atom to which they are bonded form a cycloheteroalkyl ring;
R⁶ and R⁷ are independently hydrogen, alkyl, cycloalkyl, cycloheteroalkyl, aryl or heteroaryl; and
R⁸ is —F, —Cl, —Br, —I, —R⁶, —OR⁶, —SR⁶, —NR⁶R⁷, —CF₃, —CN, —C(O)R⁶, —C(O)OR⁶, —C(O)SR⁶, —C(O)NR⁶R⁷, or aryl which is optionally substituted with one or more —F, —Cl, —Br, —I, —R⁶, —OR⁶, —SR⁶, —NR⁶R⁷, —CF₃, —CN, —C(O)R⁶, —C(O)OR⁶, —C(O)SR⁶, or —C(O)NR⁶R⁷;
wherein the center marked by * in the formula has the absolute configuration shown.

42. A compound of formula IIa: 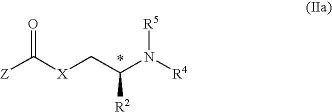
or a prodrug, salt, hydrate, solvent or N-oxide thereof,
wherein:
Z is a group of the following formula: 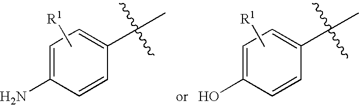
R¹ is —OR⁶, wherein R⁶ is C1-C6 alkyl;
X is —O—;
R² is methyl, isopropyl, 2-methylpropyl, 1-methylpropyl, or benzyl;
R⁴ and R⁵ are independently H, (C1-C6) alkyl or R⁴ and R⁵ together with the nitrogen atom to which they are bonded form a cycloheteroalkyl ring;
wherein the center marked by * in the formula has the absolute configuration shown.
